(19) United States
(12) Patent Application Publication
(10) Pub. No.: US 2010/0041756 A1
(43) Pub. Date: Feb. 18, 2010

(54) METHOD OF MODULATING B CELL FUNCTIONING

(76) Inventors: Michael Lionel Selley, New South Wales (AU); Julia Jane Inglis, London (GB); Richard Owen Williams, London (GB)

Correspondence Address: WILSON, SONSINI, GOODRICH & ROSATI, 650 PAGE MILL ROAD, PALO ALTO, CA, 94304-1050, US

(21) Appl. No.: 11/719,511

(22) PCT Filed: Nov. 17, 2005
(86) PCT No.: PCT/AU2005/001754

§ 371 Date: Jun. 3, 2009

Related U.S. Application Data
(60) Provisional application No. 60/628,935, filed on Nov. 17, 2004.

(51) Int. Cl. A61K 31/195; A61P 37/00

(52) U.S. Cl. 514/563

(57) ABSTRACT

The present invention relates generally to a method of modulating cellular functioning. More particularly, the present invention relates to a method of modulating B cell functioning, for example B cell proliferation, utilising an IDO-mediated tryptophan metabolite as herein defined (particular examples of such IDO-mediated tryptophan metabolites include 3-hydroxykynurenic acid, 3-hydroxyanthranilic acid, picolinic acid, quinolinic acid and tranilast). The method of the present invention is useful, inter alia, in the treatment and/or prophylaxis of conditions characterised by aberrant, unwanted or otherwise inappropriate B cell functioning such as antibody production, autoimmune conditions and B cell proliferation and neoplasias. In a related aspect, the present invention is directed to a method of therapeutically and/or prophylactically treating rheumatoid arthritis via the administration of the above-mentioned compounds.

### FIELD OF THE INVENTION

The present invention relates generally to a method of modulating cellular functioning and agents useful for same. More particularly, the present invention relates to a method of modulating B cell functioning, for example B cell proliferation, utilising a compound of formula (I). The method of the present invention is useful, inter alia, in the treatment and/or prophylaxis of conditions characterised by aberrant, unwanted or otherwise inappropriate B cell functioning such as autoimmune conditions and B cell neoplasias. In a related aspect, the present invention is directed to a method of therapeutically and/or prophylactically treating rheumatoid arthritis via the administration of a compound of formula (I).

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

“Autoimmune disease” describes the group of illnesses in which the immune system becomes misdirected and attacks one or more of the organs which it was actually designed to protect. About 75% of autoimmune disease occurs in women, most frequently during the childbearing years.

The immune system is a complicated network of cells and cell components that normally work to defend the body and eliminate infections caused by bacteria, viruses, and other invading microbes. Where a person has an autoimmune disease, the immune system mistakenly attacks self, targeting the cells, tissues, and organs of a person's own body. A collection of immune system cells and molecules at a target site is broadly referred to as inflammation.

There are many different types of autoimmune diseases, and they can each affect the body in different ways. For example, the autoimmune reaction is directed to the myelin in multiple sclerosis and the gut in Crohn's disease. Rheumatoid arthritis is characterised by the onset of an immune response to the connective tissue in the joints. In other autoimmune diseases such as systemic lupus erythematosus (lupus), affected tissues and organs may vary among individuals with the disease. One person with lupus may have affected skin and joints whereas another may have affected skin, kidney, and lungs. Ultimately, damage to certain tissues by the immune system may be permanent, as with destruction of insulin-producing cells of the pancreas in Type 1 diabetes mellitus.

The triggers for autoimmune diseases are diverse and include immunological, genetic, viral, drug-induced and hormonal factors, acting singly or in combination. At present many individual mechanisms have been identified, but how they interact with the immune network to induce such an aberrant response is likely to vary from one situation or disease condition to the next and largely has not been elucidated. Mechanisms that have been shown to eventually cause a breakdown of self tolerance include:

(1) infection of somatic tissue by viruses,
(2) development of altered self-Ags due to binding of certain drugs to cell surfaces,
(3) cross reactivity of some Abs to bacterial Ags and self-determinants,
(4) development of newly exposed Ags in the body,
(5) the influence of hormones, and
(6) breakdown in the immune network that recognizes self.

Autoimmune diseases are often chronic, requiring lifelong care and monitoring. Currently, few autoimmune diseases can be cured.

Management of the inflammatory response which is induced by an autoimmune disease can sometimes be achieved. For example, with lupus or rheumatoid arthritis immunosuppression medication can occasionally slow or stop the immune system's destruction of the targeted tissue. The drugs which are utilised in this regard include corticosteroids (prednisone), methotrexate, cyclophosphamide, azathioprine, and cyclosporin. Unfortunately, these medications also suppress the ability of the immune system to fight infection and therefore have other potentially serious side effects. However, even if a disease goes into remission, patients are rarely able to discontinue medications. The possibility that the disease may restart when medication is discontinued must therefore be balanced with the long-term side effects from treatments such as immunosuppression.

Rheumatoid arthritis is a progressive debilitating inflammatory disease of connective tissue. The most common sites affected by this disease are joints. This disease can be characterized by acute phases, followed by periods of remission. Other organs that can be involved in this systemic disorder include the lung, eye, skin, and nervous system. The course of the disease is variable, but can lead to death in active progressive forms, usually due to infection or complications of therapy. The cause of the disease is uncertain, but it has been suggested that infection with Epstein Bar Virus (EBV) may lead to activation of synovial B lymphocytes to produce an abnormal IgG Ab. The immune response to the novel Fc region of this IgG may be the production of rheumatoid factor, which can subsequently lead to immune-complex formation in the synovial fluid.

Rheumatoid arthritis usually affects the freely movable joints, the ends of the bone are covered with articular cartilage and are held together by a capsule of fibrous tissue called a joint capsule. This joint capsule is composed of an outer layer of ligaments and an inner lining of synovial membrane that secretes synovial fluid, which acts as a joint lubricant. In rheumatoid arthritis, the formation of immune complexes initiates and amplifies an inflammatory response, causing synovial membrane damage and cell lysis. Complement fragments, C3a and C5a, have anaphylatoxic and chemotactic properties. The anaphylactic activity leads to the localised release of histamine by mast cells and monocytes, producing symptoms like swelling of joints, redness and pain. Chemotactic factors can cause an influx of phagocytes to the site. These cells can also be provoked to release lysosomal enzymes into the synovial space, which furthers the inflammatory and proliferative response of the synovium.

As inflammation worsens, T and B cells can also be detected and their interaction may ensure the continued production of immunoglobulins, continuing the vicious cycle of this immune-complex syndrome. Circulating lymphocytes can enter the joint tissue from venules called the high endothelial venules. During an acute episode, the proliferating cells of the synovium can grow into the joint activity and form pannus. Pannus is composed of vascularized fibrous scar tissue that can invade the joint cavity and spread the inflammation to the articular cartilage. The hydrolytic enzymes released can erode the cartilage leading to joint destruction and other complications. There are a number of substances that can activate synoviocytes, including IL-1 and monocyte-derived tumor necrosis factor. Alternatively, the nervous system can also be involved with the release of the neuropeptide substance P, which can stimulate synoviocyte proliferation. Substance P is normally involved in the transmission of pain signals, but when released into joint tissue by sensory nerves, can stimulate the release of prostaglandins and collagenase from the rheumatoid synoviocytes. These results can also be obtained from IL-1 and TNF.

Accordingly, there is an ongoing need to develop novel means of treating diseases, such as autoimmune diseases, which are characterised by aberrant immune cell functioning. The development of therapeutic and/or prophylactic treatment regimes which provide an alternative to steroid and immunosuppression based treatments would be highly valuable when considered in light of the seriousness of the side-effects which can be associated with these current treatments.

N-[3,4-dimethoxycinnamoyl]-anthranilic acid (also known as 2-[[3-(3,4-dimethoxyphenyl)-1-oxo-2-propenyl]amino]benzoic acid, tranilast, TNL) is an anti-allergic agent originally identified as an inhibitor of mast cell degranulation [Zampini P et. al., 1983]. In work leading up to the present invention, it has been determined that IDO-mediated tryptophan metabolites or derivatives thereof, especially compounds of formula (I):

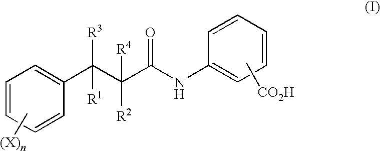

wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3; downregulate B cell functioning. These molecules are also particularly effective in treating rheumatoid arthritis.

These findings are of great significance since the elucidation of means to downregulate B cell functioning provides means for selectively regulating B cell immune responses. This has application in a variety of situations, such as the treatment of conditions which are characterised by aberrant B cell responses. Accordingly, the present invention now provides a powerful means of selectively downregulating B cell functioning in a manner which avoids the side effects associated with conventional immunosuppression, this conventional form of immunosuppression being directed to downregulating the functioning of all immune cells.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

According to one aspect of the present invention is directed to a method of down-regulating B cell functioning, said method comprising contacting said B cell with an effective amount of one or more IDO-mediated tryptophan metabolites or derivatives thereof, or pharmaceutically acceptable salts thereof.

According to another aspect of the present invention is directed to a method of downregulating B cell proliferation, said method comprising contacting said B cell with an effective amount of one or more IDO-mediated tryptophan metabolites or derivatives thereof or pharmaceutically acceptable salts thereof.

According to this preferred embodiment, there is provided a method of downregulating B cell functioning in a mammal, said method comprising administering to said mammal an effective amount of one or more IDO-mediated tryptophan metabolites or derivatives thereof or pharmaceutically acceptable salts thereof.

More particularly, there is provided a method of downregulating B cell proliferation in a mammal, said method comprising administering to said mammal an effective amount of one or more IDO-mediated tryptophan metabolites or derivatives thereof or pharmaceutically acceptable salts thereof.

According to another aspect of the present invention is directed to a method of upregulating, in a mammal, the IDO-mediated tryptophan metabolite or derivative thereof inhibited B cell functioning, said method comprising administering to said mammal an effective amount of an antagonist of an IDO-mediated tryptophan metabolite or derivative thereof or a pharmaceutically acceptable salt thereof.

According to another aspect of the present invention is directed to a method for the treatment and/or prophylaxis of a condition characterised by aberrant or unwanted B cell activity in a mammal, said method comprising administering to said mammal an effective amount of one or more IDO-mediated tryptophan metabolites or derivatives thereof or pharmaceutically acceptable salts thereof.

More particularly, there is provided a method for the treatment and/or prophylaxis of a condition characterised by aberrant or unwanted B cell functioning in a mammal, said method comprising administering to said mammal an effective amount of tranilast for a time and under conditions sufficient to downregulate said B cell functioning.

Preferably the present invention is directed to a method for the treatment and/or prophylaxis of an autoimmune condition characterised by aberrant or unwanted B cell functioning in a mammal, said method comprising administering to said mammal an effective amount of one or more IDO-mediated tryptophan metabolites or derivatives thereof or pharmaceutically acceptable salts thereof.

According to a related aspect of the present invention is directed to a method for the treatment and/or prophylaxis of inflammatory joint disease in a mammal, said method comprising administering to said mammal an effective amount of one or more IDO-mediated tryptophan metabolites or derivatives thereof or pharmaceutically acceptable salts thereof.

Yet another aspect of the present invention is directed to the use of one or more IDO-mediated tryptophan metabolites or derivatives thereof or pharmaceutically acceptable salts thereof, in the manufacture of a medicament for the treatment of a condition characterised by aberrant or unwanted B cell functioning wherein administering said compound down-regulates said B cell functioning.

Still another aspect of the present invention is directed to the use of one or more IDO-mediated tryptophan metabolites or derivatives thereof of pharmaceutically acceptable salts thereof in the manufacture of a medicament for the treatment of a condition characterised by aberrant or unwanted B cell functioning.

Yet another aspect of the present invention is directed to the use of one or more IDO-mediated tryptophan metabolites or derivatives thereof or pharmaceutically acceptable salts thereof, in the manufacture of a medicament for the treatment of inflammatory joint disease.

Yet another aspect of the present invention relates to IDO-mediated tryptophan metabolites or derivatives thereof, or pharmaceutically acceptable salts thereof or antagonists thereof, when used in the methods of the present invention.

### BRIEF DESCRIPTION OF THE DRAWINGS

FIG. 1 is a graphical representation of the amelioration of established collagen-induced arthritis (CIA) by intraperitoneal administration of tranilast. DBA/1 mice were immunised with bovine type II collagen in complete Freund's adjuvant in order to induce arthritis. After onset of clinical disease mice were randomly assigned to different treatment groups and given tranilast at 100, 200 or 400 mg/kg/day or vehicle control. Clinical scores were assessed throughout the treatment period using a clinical scoring system described in the Examples. The maximum possible score per mouse is 8. There were 7-10 mice per treatment group.

FIG. 2 is a graphical representation of the amelioration of established CIA by administration of tranilast. DBA/1 mice were immunised with bovine type II collagen in complete Freund's adjuvant in order to induce arthritis. After onset of clinical disease mice were randomly assigned to different treatment groups and given tranilast at 100, 200 or 400 mg/kg/day (i.p.) or vehicle control. Paw-swelling was measured throughout the treatment. There were 7-10 mice per treatment group.

FIG. 3 is a graphical representation of the inhibition of B cell proliferation by tranilast. B-cells were activated in vitro with LPS, and 1-100 μg/ml tranilast. Proliferation was assessed using BrdU uptake, detected by FACs analysis. LPS induced proliferation in 72% of cells. Tranilast inhibited the proliferation dose-dependently, with a maximum of 75% inhibition.

FIG. 4 is a graphical representation depicting that B cell proliferation induced by LPS and anti-CD40 is inhibited by Tranilast. B-cells were activated in vitro with LPS or anti-CD40 antibody and 6.25-100 μg/ml tranilast. Proliferation was assessed using ³[H]thymidine incorporation. LPS and anti-CD40 induced a 92- and 258-fold increase in thymidine incorporation respectively. 100 μg/ml and 50 μg/ml tranilast significantly inhibited B cell proliferation induced by LPS or anti-CD40, by 99% and 97% respectively with the 100 μg/ml dose.

FIG. 5 is a graphical representation depicting that B cell proliferation induced by LPS and anti-CD40 and anti-IgM is inhibited by tranilast. B-cells were activated in vitro with LPS or anti-CD40 antibody or anti-IgM antibody, and 12.5-100 μg/ml tranilast. Proliferation was assessed using ³[H]thymidine incorporation. LPS, anti-CD40 and anti-IgM induced 91- and 81- and 60-fold increases in thymidine incorporation respectively. 37.5 μg/ml tranilast significantly inhibited B cell proliferation induced by LPS or anti-CD 40, whilst all doses tested inhibited anti-IgM induced proliferation. Maximum inhibition was observed with 100 μg/ml tranilast of 96% (LPS), 91% (anti-CD40) and 98% (anti-IgM).

FIG. 6 is a graphical representation of the treatment of established CIA with 3,4-DAA. DBA/1 mice were immunised with type II collagen in CFA and monitored for development of arthritis. On day 1 of arthritis, mice were injected intraperitoneally with 3,4-DAA on a daily basis. Paw thickness was measured with callipers. The clinical scoring system was as follows: 0=normal, 1=slight swelling and/or erythema, and 2=pronounced oedematous swelling. Each limb was graded, giving a maximum score of 8 per mouse. Histological assessment of arthritis was carried out on haematoxylin and eosin stained sections using a scoring system as follows: 0, normal; 1, minimal synovitis without cartilage/bone erosion; 2, synovitis with some marginal erosion but joint architecture maintained; 3, severe synovitis and erosion with loss of normal joint architecture. There were 14 mice/group (data pooled from two separate experiments). *, P<0.05 (compared to control group).

FIG. 7 is a graphical representation depicting that treatment with 3,4-DAA leads to increased IL-10 levels in vivo. Mice with established CIA were treated with 3,4-DAA or vehicle (n=7) for 10 days (see FIG. 6), then bled. IL-10 in the sera was measured by ELISA.

FIG. 8 is a graphical representation depicting that mice with established CIA were treated for 10 days with 3,4-DAA or vehicle control. Mice were then killed and draining (inguinal) lymph node cells were cultured for 72 h in the absence or presence of type II collagen. IFN-γ and IL-5 production was measured by ELISA and was found to be significantly reduced in the mice given 3,4-DAA at 400 mg/kg. However, on restimulation with collagen, differences between the groups were not significant, indicating that the ability of the T cells to respond to antigenic stimulation returned to normal in the absence of the drug.

FIG. 9 is a graphical representation depicting the relapse of arthritis 4 days after cessation of therapy. Mice with established CIA (n=6) were treated with 3,4-DAA (400 mg/kg/day) from days 1 to 5 of arthritis and clinical severity of arthritis was monitored up to day 12. Arthritis is seen to relapse at around day 9.

FIG. 10 is a graphical representation depicting that 3,4-DAA and 3-HAA inhibit B and T cell proliferation in vitro. Purified B and T cells were stimulated for 72 h with anti-CD40 (a), or anti-CD3/anti-CD28 (b) respectively, in the presence of varying doses of 3,4-DAA, or 3-HAA. Both 3,4-DAA, and 3-HAA dose-dependently inhibited B and T cell proliferation, assessed by 3H-thymidine incorporation. Both 3,4-DAA and 3-HAA therapy dose-dependently reduced IFN-γ production by T-cells (c). 3,4-DAA dose-dependently inhibited IL-10 and IL-5 production (d, e), whilst 3-HAA increased IL-10 and IL-5 production by T-cells.

### DETAILED DESCRIPTION OF THE INVENTION

The present invention is predicated, in part, on the surprising determination that IDO-mediated tryptophan metabolites or derivatives thereof, especially compounds of formula (I), downregulate B cell functioning, in particular B cell proliferation. Consistent with these findings, and in a related aspect, it has also been determined that IDO-mediated tryptophan metabolites or derivatives thereof, especially compounds of formula (I), are particularly effective in downregulating the autoimmune response associated with rheumatoid arthritis. These findings have now permitted the rational design of means for therapeutically or prophylactically treating conditions which are characterised by aberrant or unwanted B cell functioning. Examples of such conditions include autoimmune conditions such as rheumatoid arthritis.

Accordingly, one aspect of the present invention is directed to a method of down-regulating B cell functioning, said method comprising contacting said B cell with an effective amount of one or more IDO-mediated tryptophan metabolites or derivatives thereof or pharmaceutically acceptable salts thereof.

Reference to “IDO-mediated tryptophan metabolites” should be understood as a reference to any molecule which is generated pursuant to the metabolism of tryptophan via the IDO enzyme system. Examples of such metabolites include, but are not limited to, 3-Hydroxykynurenic acid (3-HKA), 3-Hydroxyanthranilic acid (3-HAA), picolinic acid (PA), and quinolinic acid (QA). The present invention should also be understood to extend to the use of derivatives of IDO-mediated tryptophan metabolites, such as tranilast. N-[3,4-dimethoxycinnamoyl]-anthranilic acid (also known as 2-[[3-(3,4-dimethoxyphenyl)-1-oxo-2-propenyl]amino]benzoic acid, tranilast, TNL) is an anti-allergic agent originally identified as an inhibitor of mast cell degranulation (Zampini P et al., 1983). In accordance with the present invention, it has been determined that this molecule, which is a synthetic derivative of 3-HAA, functions to downregulate B cell functioning.

In one preferred embodiment, the IDO-mediated tryptophan metabolite or derivative thereof is a compound of formula (I):

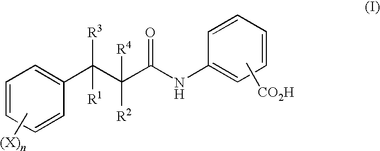

wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3 or a pharmaceutically acceptable salt thereof.

The carboxyl group in the compound of formula (I) may be in the 2-, 3- or 4-position of the aromatic ring. Preferably the carboxyl group is in the 2-position.

Preferably at least one of R¹ and R² is a hydrogen atom. More preferably, both of R¹ and R² are hydrogen atoms.

Preferably R³ and R⁴ taken together form a chemical bond. Such compounds having an unsaturated bond may be in the form of E or Z geometric isomers.

Preferably n is 1 or 2 and each X, which may be the same or different, is selected from halogen, C₁-C₄ alkyl or C₁-C₄alkoxy. Preferably X is selected from halogen and C₁-C₄alkoxy. More preferably, n is 2 and both X are selected from C₁-C₄alkoxy, especially when both X are methoxy.

Particularly preferred compounds of formula (I) useful in the invention are those of formula (II):

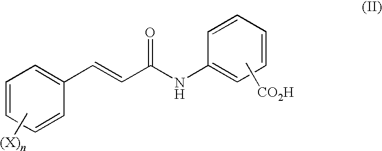

wherein X and n are defined in formula (I).

Examples of compounds of formula (II) include
2-[[3-(2-methylphenyl)-1-oxo-2-propenyl]amino]benzoic acid;
2-[[3-(3-methylphenyl)-1-oxo-2-propenyl]amino]benzoic acid;
2-[[3-(4-methylphenyl)-1-oxo-2-propenyl]amino]benzoic acid;
2-[[3-(2-ethylphenyl)-1-oxo-2-propenyl]amino]benzoic acid;
2-[[3-(3-ethylphenyl)-1-oxo-2-propenyl]amino]benzoic acid;
2-[[3-(4-ethylphenyl)-1-oxo-2-propenyl]amino]benzoic acid;
2-[[3-(2-propylphenyl)-1-oxo-2-propenyl]amino]benzoic acid;
2-[[3-(3-propylphenyl)-1-oxo-2-propenyl]amino]benzoic acid;
2-[[3-(4-propylphenyl)-1-oxo-2-propenyl]amino]benzoic acid;
2-[[3-(2-hydroxyphenyl)-1-oxo-2-propenyl]amino]benzoic acid;
2-[[3-(3-hydroxyphenyl)-1-oxo-2-propenyl]amino]benzoic acid;
2-[[3-(4-hydroxyphenyl)-1-oxo-2-propenyl]amino]benzoic acid;
2-[[3-(2-chlorophenyl)-1-oxo-2-propenyl]amino]benzoic acid;
2-[[3-(3-chlorophenyl)-1-oxo-2-propenyl]amino]benzoic acid;
2-[[3-(4-chlorophenyl)-1-oxo-2-propenyl]amino]benzoic acid;
2-[[3-(2-fluorophenyl)-1-oxo-2-propenyl]amino]benzoic acid;
2-[[3-(3-fluorophenyl)-1-oxo-2-propenyl]amino]benzoic acid;
2-[[3-(4-fluorophenyl)-1-oxo-2-propenyl]amino]benzoic acid;
2-[[3-(2-bromophenyl)-1-oxo-2-propenyl]amino]benzoic acid;
2-[[3-(3-bromophenyl)-1-oxo-2-propenyl]amino]benzoic acid;
2-[[3-(4-bromophenyl)-1-oxo-2-propenyl]amino]benzoic acid;
2-[[3-(2,3-dimethoxyphenyl)-1-oxo-2-propenyl]amino]benzoic acid;
2-[[3-(3,4-dimethoxyphenyl)-1-oxo-2-propenyl]amino]benzoic acid;
2-[[3-(2,4-dimethoxyphenyl)-1-oxo-2-propenyl]amino]benzoic acid;
2-[[3-(2,3-dimethylphenyl)-1-oxo-2-propenyl]amino]benzoic acid;
2-[[3-(3,4-dimethylphenyl)-1-oxo-2-propenyl]amino]benzoic acid;
2-[[3-(2,4-dimethylphenyl)-1-oxo-2-propenyl]amino]benzoic acid;
2-[[3-(2,3-diethoxyphenyl)-1-oxo-2-propenyl]amino]benzoic acid;
2-[[3-(3,4-diethoxyphenyl)-1-oxo-2-propenyl]amino]benzoic acid;
2-[[3-(2,4-diethoxyphenyl)-1-oxo-2-propenyl]amino]benzoic acid;
2-[[3-(2,3-dipropoxyphenyl)-1-oxo-2-propenyl]amino]benzoic acid;
2-[[3-(3,4-dipropoxyphenyl)-1-oxo-2-propenyl]amino]benzoic acid;
2-[[3-(2,4-dipropoxyphenyl)-1-oxo-2-propenyl]amino]benzoic acid;
2-[[3-(2,3-diethylphenyl)-1-oxo-2-propenyl]amino]benzoic acid;
2-[[3-(3,4-diethylphenyl)-1-oxo-2-propenyl]amino]benzoic acid;
2-[[3-(2,4-diethylphenyl)-1-oxo-2-propenyl]amino]benzoic acid;
2-[[3-(2,3-dipropylphenyl)-1-oxo-2-propenyl]amino]benzoic acid;
2-[[3-(3,4-dipropylphenyl)-1-oxo-2-propenyl]amino]benzoic acid;
2-[[3-(2,4-dipropylphenyl)-1-oxo-2-propenyl]amino]benzoic acid;
2-[[3-(2-methoxy-3-methylphenyl)-1-oxo-2-propenyl]amino]benzoic acid;
2-[[3-(3-methoxy-4-methylphenyl)-1-oxo-2-propenyl]amino]benzoic acid;
2-[[3-(2-methoxy-3-methylphenyl)-1-oxo-2-propenyl]amino]benzoic acid;
2-[[3-(2-methoxy-4-methylphenyl)-1-oxo-2-propenyl]amino]benzoic acid;
2-[[3-(2-methoxy-3-chlorophenyl)-1-oxo-2-propenyl]amino]benzoic acid;
2-[[3-(3-methoxy-4-chlorophenyl)-1-oxo-2-propenyl]amino]benzoic acid;
2-[[3-(2-methoxy-3-chlorophenyl)-1-oxo-2-propenyl]amino]benzoic acid;
2-[[3-(2-methoxy-4-chlorophenyl)-1-oxo-2-propenyl]amino]benzoic acid;
2-[[3-(2-methoxy-3-hydroxyphenyl)-1-oxo-2-propenyl]amino]benzoic acid;
2-[[3-(3-methoxy-4-hydroxyphenyl)-1-oxo-2-propenyl]amino]benzoic acid;
2-[[3-(2-methoxy-3-hydroxyphenyl)-1-oxo-2-propenyl]amino]benzoic acid;
2-[[3-(2-methoxy-4-hydroxyphenyl)-1-oxo-2-propenyl]amino]benzoic acid;
2-[[3-(3,4-trimethylenephenyl)-1-oxo-2-propenyl]amino]benzoic acid;
2-[[3-(2,3-trimethylenephenyl)-1-oxo-2-propenyl]amino]benzoic acid;
2-[[3-(3,4-methylenedioxyphenyl)-1-oxo-2-propenyl]amino]benzoic acid; and
2-[[3-(3,4-ethylenedioxyphenyl)-1-oxo-2-propenyl]amino]benzoic acid.

A particularly preferred compound of formula (II) for use in the invention is 2-[[3-(3,4-dimethoxyphenyl)-1-oxo-2-propenyl]amino]benzoic acid (tranilast, TNL).

In another preferred embodiment, the IDO-mediated tryptophan metabolite or derivative thereof is a compound of formula (III):

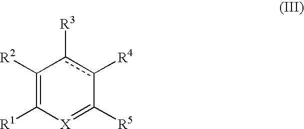

wherein
X is selected from N and CR⁶; [symbol]
represents a single or double bond;
R¹ is selected from H, C₁₋₄alkyl, OH, C₁₋₄alkoxy, halo, CO₂H and CO₂C₁₋₄alkyl;
R² is selected from H, C₁₋₄alkyl, OH, C₁₋₄alkoxy, halo, or R¹ and R² together form an optionally substituted fused phenyl ring;
R³ is selected from H, C₁₋₄alkyl, OH, C₁₋₄alkoxy and halo;
R⁴ is selected from H, C₁₋₄alkyl, C₂₋₄alkenyl, OH, C₁₋₄alkoxy, CO₂H, CO₂C₁₋₄alkyl and

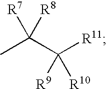

R⁵ is selected from C₁₋₄alkyl, OH, C₁₋₄alkoxy, halo, CO₂H, CO₂C₁₋₄alkyl, NH₂ and NHR¹²;
R⁶ is selected from H, C₁₋₄alkyl, OH and C₁₋₄alkoxy;
R⁷, R⁸, R⁹ and R¹⁰ are each independently H and C₁₋₄alkyl or R⁷ and R⁸ together form an oxo group or R⁷ and R⁹ form a bond;
R¹¹ is selected from CH(CO₂H)NH₂, CH(CO₂C₁₋₄alkyl)NH₂, C(O)CO₂H, C(O)CO₂C₁₋₄alkyl, C(O)H, CO₂H, CO₂C₁₋₄alkyl, C(O)NH₂, C(O)NHR¹³, CH₂NH₂, CH₂NHC₁₋₄alkyl and CH₂N(C₁₋₄alkyl)₂;
R¹² is selected from H, C₁₋₄alkyl and C(O)H; and
R¹³ is H, C₁₋₄alkyl and optionally substituted phenyl, wherein optionally substituted phenyl is optionally substituted with one or more, C₁₋₄alkyl, OH, C₁₋₄alkoxy, CO₂H, CO₂C₁₋₄alkyl, halo, NH₂, NHC₁₋₄alkyl and N(C₁₋₄alkyl)₂ or a pharmaceutically acceptable salt thereof.

Preferably, said compound of formula (III) is 3-HKA, 3HAA, PA or QA.

As used herein, the term “C₁-C₄alkyl” refers to linear or branched alkyl groups having 1 to 4 carbon atoms. Examples of such groups include methyl, ethyl, n-propyl, isopropyl, n-butyl, sec-butyl and tert-butyl.

As used herein, the term “C₂-C₄alkenyl” refers to linear or branched hydrocarbon chains having 2 to 4 carbon atoms and one or two double bonds. Examples of such groups include vinyl, propenyl, butenyl and butadienyl.

As used herein, the term “C₁-C₄alkoxy” refers to hydroxy groups substituted with linear or branched alkyl groups having 1 to 4 carbon atoms. Examples of such groups include methoxy, ethoxy, n-propoxy, isopropoxy, n-butoxy, sec-butoxy and tert-butoxy.

As used herein, the term “halogen” or “halo” refers to fluoro, chloro or bromo atoms.

Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benzenesulphonic, salicyclic sulphanilic, aspartic, glutamic, edetic, stearic, palmitic, oleic, lauric, pantothenic, tannic, ascorbic and valeric acids.

Base salts include, but are not limited to, those formed with pharmaceutically acceptable cations, such as sodium, potassium, lithium, calcium, magnesium, ammonium and alkylammonium.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

Compounds of formula (I) and their pharmaceutically acceptable salts are known and may be prepared by methods known in the art, see U.S. Pat. No. 3,940,422 the contents of which are incorporated herein by reference.

Compounds of formula (III) such as 3-hydroxyanthranilic acid, quinolinic acid, picolinic acid, kynurenine, xanthurenic acid and kynurenic acid may be purchased from speciality chemical companies. Alternatively, compounds of formula (III) may be synthesised using synthetic techniques known to those skilled in the art. For example 3-methoxyanthranilic acid and 8-methoxykynurenic acid can be prepared from 3-hydroxyanthranilic acid and xanthurenic acid respectively by methylation of a hydroxy, for example, using diazomethane. Alternatively, compounds of formula (III) may be prepared by enzymatic transformation, for example 3-hydroxy-kynurenic acid may be prepared from kynurenic acid by oxidation with kynurenic acid hydroxylase (EC 1.14.992) and then rearomatisation with kynurenate-7,8-dihydrodiol dehydrogenase (EC 1.3.1.18).

It will also be recognised that some compounds of formulae (I), (II) and (III) may possess asymmetric centres and are therefore capable of existing in more than one stereoisomeric form. The invention thus also relates to compounds in substantially pure isomeric form at one or more asymmetric centres eg., greater than about 90% ee, such as about 95% or 97% ee or greater than 99% ee, as well as mixtures, including racemic mixtures, thereof. Such isomers may be prepared by asymmetric synthesis, for example using chiral intermediates, or by chiral resolution.

Without limiting the present invention to any one theory or mode of action, the compounds of formula (I) are orally active anti-allergic compounds. A particularly preferred compound of the invention is known by either of the chemical names N-[3,4-dimethoxycinnamoyl]-anthranilic acid or 2-[[3-(3,4-dimethoxyphenyl)-1-oxo-2-propenyl]amino]benzoic acid and may also be referred to as Tranilast. Still further, it is known by the chemical formula C₁₈H₁₇NO₅ and by the trade name Rizaben. The structure of N-[3,4-dimethoxycinnamoyl]-anthranilic acid is depicted below:

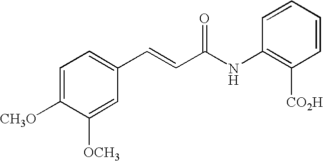

Reference to a “B cell” (also known as a “B lymphocyte”) should be understood as a reference to the immune cells which express a cell surface immunoglobulin molecule and which, upon activation, terminally differentiate into cells which secrete antibody. Accordingly, this includes, for example, convention at B cells, CD5 B cells (also known as B-1 cells and transitional CD5 B cells). Reference to “B cell” should also be understood to encompass reference to B cell mutants. “Mutants” include, but are not limited to, B cells which have been naturally or non-naturally modified, such as cells which are genetically modified. Reference to “B cells” should also be understood to extend to B cells which exhibit commitment to the B cell image. These cells may be at any differentiative stage of development and therefore may not necessarily express a surface immunoglobulin molecule. B cell commitment may be characterised by the onset of immunoglobulin gene re-arrangement or it may correspond to an earlier stage of commitment which is characterised by some other phenotypic or functional characteristic such as the cell surface expression of CD45R, MHCII, CD10, CD19 and CD38. Examples of B cells at various stages of differentiation include early B cell progenitors, early pro-B cells, late pro-B cells, pre-B cells, immature B cells, mature B cells and plasma cells.

Reference to B cell “functioning” should be understood as a reference to any one or more of the functional activities which a B cell, at any differentiative stage of development, is capable of performing. This includes, for example, proliferation, differentiation, immunoglobulin gene rearrangement, immunoglobulin synthesis and secretion and antigen presentation. Preferably, the subject functioning is B cell proliferation. In this regard, by preventing expansion of a B cell population, there is a direct impact, and effectively a downregulation, of B cell functional end points such as antigen presentation and immunoglobulin secretion. Accordingly, the modulation of B cell numbers provides a highly valuable and effective means of modulating the extent and effectiveness of B cell related antigen presentation or antibody secretion. In another preferred embodiment, said functioning is antibody production, irrespective of any concurrent change to B cell proliferation.

According to another aspect of the present invention is directed to a method of downregulating B cell proliferation, said method comprising contacting said B cell with an effective amount of one or more IDO-mediated tryptophan metabolites or derivatives thereof or pharmaceutically acceptable salts thereof.

Preferred IDO-mediated tryptophan metabolites or derivatives thereof are those compounds of formulae (I), (II) and (III) described above, especially tranilast, 3-HKA, 3-HAA, PA and QA.

It should be understood that the cell which is the subject of modulation in accordance with the method of the invention may be an isolated B cell or a B cell which forms part of a group of cells, such as an isolated tissue. The B cell may also be localised in a mammal, that is it is not isolated, therefore requiring the subject method to be performed in vivo. Where the subject cell is one of a group of cells or a tissue, either isolated or not, the subject method may modulate the functioning of all the B cells in that group or just a subgroup of B cells in that group. Similarly, in the context of the modulation of the biological functioning or development of a mammal, it should be understood that the subject modulation may be achieved in the context of modulating B cell functioning either systemically or in a localised manner. Still further, irrespective of which means is employed, the cellular impact of the change in B cell functioning may occur in the context of either all cells or just a subgroup of cells within the relevant environment.

Reference to “modulating” should be understood as a reference to upregulating or downregulating the functional activity of a mammalian B cell. Reference to “downregulation” in this context should be understood as a reference to preventing, reducing (eg. slowing) or otherwise inhibiting one or more aspects of said activity while reference to “upregulating” in this context should be understood to have the converse meaning.

It should be understood that the B cell which is treated according to the method of the present invention may be located ex vivo or in vivo. By “ex vivo” is meant that the cell has been removed from the body of a subject wherein the modulation of its activity will be initiated in vitro. For example, the cell may be a B cell which is to be used as a model for studying any one or more aspects of the pathogenesis of autoimmune conditions which are characterised by aberrant B cell activity. In a preferred embodiment, the subject cell is located in vivo.

According to this preferred embodiment, there is provided a method of downregulating B cell functioning in a mammal, said method comprising administering to said mammal an effective amount of one or more IDO-mediated tryptophan metabolites or derivatives thereof or pharmaceutically acceptable salts thereof.

More particularly, there is provided a method of downregulating B cell proliferation in a mammal, said method comprising administering to said mammal an effective amount of one or more IDO-mediated tryptophan metabolites or derivatives thereof or pharmaceutically acceptable salts thereof.

In preferred embodiments of these methods, the IDO-mediated tryptophan metabolite or derivative thereof is a compound of formulae (I), (II) or (III) or a pharmaceutically acceptable salt thereof, in particular tranilast, 3-HKA, 3-HAA, PA and QA or pharmaceutically acceptable salts thereof.

The term “mammal” as used herein includes humans, primates, livestock animals (eg. sheep, pigs, cattle, horses, donkeys), laboratory test animals (eg. mice, rabbits, rats, guinea pigs), companion animals (eg. dogs, cats) and captive wild animals (eg. foxes, kangaroos, deer). Preferably, the mammal is human or a laboratory test animal. Even more preferably, the mammal is a human.

Although the preferred method is to downregulate B cell functioning, it may also be desired to induce the upregulation of this activity in certain circumstances. For example, in certain conditions the administration of one or more IDO-mediated tryptophan metabolites or derivatives thereof may be an appropriate systemic therapy. Accordingly, a side effect of such therapy may well be unwanted downregulation of B cell functioning in certain cell groups or at certain tissue sites. To the extent that it is not possible to rectify this situation by ceasing administration of the one or more IDO-mediated tryptophan metabolites or derivatives thereof, it may be desirable to administer, (in a site directed manner, for example) an antagonistic agent of the one or more IDO-mediated tryptophan metabolites or derivatives thereof. In another example, therapy with one or more IDO-mediated tryptophan metabolites or derivatives thereof may necessitate the use of antagonists of the one or more IDO-mediated tryptophan metabolites or derivatives thereof in order to inhibit the functioning of the compound which has been introduced to a mammal but which functional activity is required to be slowed or stopped. Reference to “one or more IDO-mediated tryptophan metabolites or derivatives thereof inhibited B cell functioning” should therefore be understood to mean that at least some of the B cell functioning of the mammal exhibits inhibited, slowed or otherwise retarded functioning due to the effects of the one or more IDO-mediated tryptophan metabolites or derivatives thereof or a pharmaceutically acceptable salt thereof.

Accordingly, another aspect of the present invention is directed to a method of upregulating, in a mammal, IDO-mediated tryptophan metabolite inhibited B cell functioning, said method comprising administering to said mammal an effective amount of an antagonist of a IDO-mediated tryptophan metabolite or derivative thereof or a pharmaceutically acceptable salt thereof.

Reference to “antagonist of an IDO-mediated tryptophan metabolite or derivative thereof or a pharmaceutically acceptable salt thereof” should be understood as a reference to any proteinaceous or non-proteinaceous molecule which directly or indirectly inhibits, retards or otherwise downregulates the cell functioning inhibitory activity of the IDO-mediated tryptophan metabolites or derivatives thereof or pharmaceutically acceptable salts thereof. Identification of antagonists suitable for use in the present invention can be routinely achieved utilising methods well known to those skilled in the art.

A further aspect of the present invention relates to the use of the invention in relation to the treatment and/or prophylaxis of disease conditions or other unwanted conditions or a predisposition to the onset of such a condition. More particularly, the present invention is directed to the treatment of disease conditions characterised by aberrant or unwanted B cell functioning, such as aberrant or unwanted B cell proliferation. Without limiting the present invention to any one theory or mode of action, conditions which may be treated in accordance with the method of the present invention include, but are not limited, autoimmune conditions, acute and chronic organ rejection and B cell neoplasias.

In the context of autoimmune disease, conditions which may be treated in accordance with the method of the present invention include but are not limited to:

### (i) Rheumatoid Arthritis

It has been shown that B cells are important in synovial inflammation and have potential as therapeutic targets (Takemura et al., J. Immunol. 2001; 167(8): 4710-4718; Silverman et al, Arthritis Res. Ther. 2003; 5(Suppl 4): S1-S6; Looney et al., Curr. Opin. Rheumatol. 2004; 16: 180-185; Oligino et al., Arthritis Res. Ther. 2003; 5(Suppl 4): S7-S11; Silverman et al., Arthritis Rheum. 2003; 48(6): 1484-1492; Gorman et al., Arthritis Res. Ther. 2003; 5 (Suppl 4): S17-S21). Bone marrow participates in rheumatoid arthritis by generating B cell-rich lesion which induce endosteal bone formation (Hayer et al., Bone Miner. Res. 2004, 19(6):990-998). The contribution of B cells in rheumatoid arthritis has been validated by data from clinical trials indicating that B cell depletion with rituximab is highly therapeutic (Edwards et al., N. Engl. J. Med. 2004; 350 (25): 2572-2581).

### (ii) Multiple Sclerosis

In the past research has largely focused on the contribution of T cells in multiple sclerosis but recent studies are revealing the potential role of B cells in the disease process (Archelos et al., Ann. Neurol. 2000; 47(6): 694-706; Iglesias et al., Glia 2001; 36(2): 220-234; Hemmer et al., Nat. Rev. Neurosci. 2002; 3(4): 291-301; Hemmer et al., Curr. Opin. Neurol. 2002; 15(3): 227-231; Qin et al., Int. MS J. 2003; 10(4): 110-120; Burgoon et al., Front. Biosci. 2004; 1(9): 786-796; Alter et al., J. Immunol. 2003; 170: 4497-4505). The B cell mediated immune response is an early event of the inflammatory reaction in the central nervous system in multiple sclerosis (Qin et al., Lab. Invest. 2003; 83(7): 1081-1088; Haubold et al., Ann. Neurol. 2004, 56(1):97-107). The contribution of B cells may be mainly through demyelination (Svensson et al., Eur. J. Immunol. 2002; 32(7): 1939-1946). Tranilast may inhibit both the inflammatory response and demyelination in multiple sclerosis.

(iii) Systemic Lupus Erythematosus

B cells play a central role in the pathogenesis of systemic lupus erythematosus (SLE) (Looney et al. 2004, supra; Chan et al., Immunol. Rev. 1999; 169: 107-121; Looney et al., Arthritis. Rheum. 2004; 50(8): 2580-2589; Anolik et al., Curr. Opin. Rheumatol. 2004; 16(5): 505-512; Looney et al., Lupus 2004; 13(5): 381-390; Baker et al., Autoimmun. Rev. 2004; 3(5): 368-375; Higuchi et al., J. Immunol. 2002; 168(1): 9-12; Desai-Mehta et al., J. Clin. Invest. 1996; 97(9): 2063-2073). An antibody-independent role of B cells has been demonstrated in murine lupus (Chan et al., J. Immunol. 1999; 163(7): 3592-3596; Chan et al., J. Exp. Med. 1999; 189(10): 1639-1648). This has been confirmed by a significant improvement in disease activity in patients treated with rituximab even in the absence of substantial serologic responses (Looney et al., 2004, supra). The successful treatment of SLE with rituximab demonstrates the value in targeting B cells in this disease (Looney et al., 2004, supra).

### (iv) Psoriatic Arthritis

There is evidence that antigen-activated B cells participate in the development of chronic synovitis in psoriatic arthritis (Gerhard et al., Z. Rheumatol. 2002, 61(6):718-727).

### (v) Inflammatory Bowel Disease

The two major forms of inflammatory bowel disease (IBD) are Crohn's disease and ulcerative colitis. The presence of circulating antibodies to colonic epithelial cells has been reported in Crohn's disease and ulcerative colitis (Hibi et al., Clin. Exp. Immunol. 1983; 54(1): 163-168; Takahashi et al., J. Clin. Invest. 1985; 76(1): 311-318; Sadlack et al., Cell 1993; 75(2): 253-261). There is evidence that the pathogenesis of IBD may be triggered by a primarily B cell mechanism through the ectopic expression of the CD40 ligand (CD40L) on B cells (Kawamura et al., J. Immunol. 2004; 172(10): 6388-6397). A similar ectopic expression of CD40L in B cells can induce a lupus-like disease and there is an increased expression of CD40L by B cells in SLE (Desai-Mehta et al., 1996, supra).

Several reports have described a relationship between IBD and SLE supporting the idea that both IBD and SLE may be triggered by primarily dysregulated B cells (Ishikawa et al., J Dermatol. 1995; 22(4): 289-291; Kritikos et al., Eur. J. Gastroenterol. Hepatol. 1998; 10(5): 437-439). It has also been shown that B cells may play an important role in the development of inflammation in a murine model of Crohn's disease by inhibiting regulatory T cells (Olson et al., J. Clin. Invest. 2004; 114(3): 389-398).

### (vi) Type 1 Diabetes

B cells play a critical role as antigen-presenting cells in the development of T cell-mediated autoimmune type 1 diabetes in the nonobese diabetic mouse (Noorchashm et al., Diabetes 1997; 46(6): 941-946; Noorchashm et al., J. Immunol. 1999; 163(2): 743-750; Greeley et al., J. Immunol. 2001; 167(8): 4351-4357; Akashi et al., Int. Immunol. 1997; 9(8): 1159-1164; Serreze et al., J. Exp. Med. 1996; 184(5): 2049-2053; Serreze et al., J. Immunol. 1998; 161(8): 3912-3918; Chiu et al., Diabetes 2001; 50(4): 763-770; Silveira et al, Eur. J. Immunol. 2002; 32(12): 3657-3666; Serreze et al., Curr. Dir. Autoimmun. 2003; 6: 212-227; Silveira et al., J. Immunol. 2004; 172(8): 5086-5094). This collaboration between T cells and B cells in autoimmune diabetes indicates that a drug such as tranilast, which can downregulate B cell functioning will be efficacious. There is also evidence that interventions directed at B cells may be useful in the later stages of the disease (Kendall et al., Euro J. Immunol. 2004; 34(9): 2387-2). This may be important in the treatment of human type 1 diabetes in which early diagnosis and appropriate preventative measures are difficult.

(vii) Psoriasis

Psoriasis is now considered to be a T cell-mediated disease (Morel et al., J Autoimmun. 1992; 5(4): 465-477; Bachelez et al., J Autoimmun. 1998; 11(1): 53-62; Boyman et al., J. Exp. Med. 2004; 199(5): 731-736). Increased B cell infiltration has been reported in the lesional tissue of patients with non-arthritic psoriasis (Griffiths C. E. J. Eur. Acad. Dermatol. Venerol. 2003; 17(Suppl 2): 1-5). There are associations between psoriasis and Crohn's disease (Sarwal et al., N. Engl. J. Med. 2003; 349(2): 125-138). B cells are involved in the pathology of Crohn's disease and may contribute to the development of psoriasis (Olson et al., 2004, supra).

(viii) Graves' Disease, Hashimoto's Thyroiditis and Autoimmune Thyroiditis

B cells are involved in the pathology of Grave's disease and autoimmune thyrioditis (Hasselbalch, Immun. Lett. 2003; 88(1): 85-86; Nielsen et al., Eur. J. Immunol., 2004; 34(1): 263-272).

### (ix) Systemic Sclerosis

There is a disturbed B cell homeostasis and increased memory B cell hyperactivity in scleroderma indicating that B cells may be a target in the treatment of scleroderma (Sato et al., Arthritis Rheum. 2004; 50(6): 1918-1927; Asano et al., Am. J. Pathol. 2004; 165(2): 641-650).

### (x) Chronic Immune Thrombocytopenic Purpura

The depletion of B cells is useful in the treatment of chronic immune thrombocytopenic purpura (Stasi et al., Blood 2001; 98(4): 952-957; Cooper et al., Br. J. Haematol. 2004; 125(2): 232-239; Ahmad et al., Am. J. Hematol. 2004; 77(2): 171-176).

### (xi) Other Autoimmune Disorders

B cell depletion therapy is also effective in Sjogren's syndrome autoimmune polyneuropathy (Levine and Pestronk, Neurology 1999; 52(8): 1701-1704), Wegener's granulomatosis (Specks et al., Arthritis Rheum. 2001; 44(12): 2836-2840), cold agglutinin disease associated with indolent lymphoma (Cohen et al., Leuk. Lymphoma 2001; 42(6): 1405-1408; Berensen et al., Blood 2004; 103(8): 2925-2928), idiopathic membranous neuropathy (Ruggenenti et al., J. Am. Soc. Nephrol. 2003; 14(7): 1851-1857), type II mixed cryoglobulinaemia (Zaja et al. Blood 2003; 101(10): 3827-3834), acquired factor VIII inhibitors (Wiestner et al. Blood 2002; 100(9): 3426-3428; Stasi et al., Blood 2004; 103(12): 4424-4428), fludarabine-associated immune thrombocytopenic purpura (Hegde et al., Blood 2002; 100(6): 2260-2262), refractory dermatomyositis (Levine, Arthritis Rheum. 2002; 46 Suppl. 9): S488, pemphigus vulgaris (Dupuy et al., Arch. Dermatol. 2004; 140(1): 91-96) and myasthenia gravis (Zaja et al., Neurology 2000; 55(7): 1062-1063; Wylam et al., J Pediatr. 2003; 143 (5): 674-677; Gajra et al., Am. J. Hematol 2004; 77(2): 196-197).

Non-autoimmune conditions which may be treated in accordance with the method of the present invention include:

### (i) Chronic Transplant Rejection

Since a major component of chronic rejection is antibody mediated, drugs which inhibit B cells may reduce the production of antibodies (Pescovitz M. D. 2004, supra). Mycophenolate mofetil and sirolimus inhibit B cell proliferation and reduce antibody formation to a neoantigen in transplant recipients (Kimball et al., Transplantation 1995; 60(12): 1379-1383; Pescovitz et al., Am. J. Transplant. 2003; 3(4): 497-500).

### (ii) B Cell Lymphomas

The anti-CD20 monoclonal antibody rituximab is standard therapy in the treatment of non-Hodgkin's lymphoma and has been used in a number of other B cell malignancies, including indolent and follicular lymphoma, mantle cell lymphoma, chronic lymphocytic leukaemia, small lymphocytic lymphoma, multiple myeloma, primary cutaneous B cell lymphomas, acute lymphocytic leukaemia, Burkitt's lymphoma, HIV-associated lymphoma, primary CNS lymphoma, post-transplant lymphoproliferative disorder and Hodgkin's disease (Boye et al., Ann. Oncol. 2003; 14(4): 520-535; Avivi et al., Br. J. Cancer. 2003; 89(8): 1389-1394; Rastetter et al., Annu. Rev. Med. 2004; 55: 477-503.). This indicates a role for tranilast in the treatment of lymphomas alone and in combination with standard chemotherapy.

(iii) Graft-Versus Host Disease (GVHD)

GVHD is characterised by a pathogenic role of B cells in this disease (Ratanatharathorn et al., Biol. Blood Marrow Transplant 2003; 9(8): 505-511).

### (iv) Acute Transplant Rejection

Infiltrating B cells play a pivotal role in acute transplant rejection (Sarwal et al., 2003, supra; Krukemeyer et al., Transplantation 2004; 78(1): 65-70). B cell MHC class II-mediated antigen presentation contributes to the pathogenesis of acute allograft rejection (Akashi et al., 1997, supra).

Accordingly, another aspect of the present invention is directed to a method for the treatment and/or prophylaxis of a condition characterised by aberrant or unwanted B cell activity in a mammal, said method comprising administering to said mammal an effective amount of one or more IDO-mediated tryptophan metabolites or derivatives thereof or pharmaceutically acceptable salts thereof.

Preferably, the IDO-mediated tryptophan metabolite or derivative thereof is a compound of formula (I), (II) or (III), especially tranilast, 3-HKA, 3-HAA, PA or QA.

More particularly, there is provided a method for the treatment and/or prophylaxis of a condition characterised by aberrant or unwanted B cell functioning in a mammal, said method comprising administering to said mammal an effective amount of tranilast for a time and under conditions sufficient to downregulate said B cell functioning.

Preferably, said B cell functioning is B cell proliferation.

Reference to a condition characterised by “aberrant or unwanted” B cell functioning should be understood as a reference to B cell functioning which is either not normal or which is physiologically normal but is inappropriate in that it is unwanted. Examples of such conditions include, but are not limited to, autoimmune conditions such as rheumatoid arthritis, multiple sclerosis, Crohn's disease, inflammatory bowel disease, type I diabetes, psoriasis, Graves' disease, autoimmune thyroiditis, systemic sclerosis, chronic immune thrombocytopenic purpura, autoimmune haemolytic anaemia, autoimmune polyneuropathy, Wegener's granulomatosis, cold agglutinin disease associated with indolent lymphoma, idiopathic membranous neuropathy, type II mixed cryoglobulinaemia, acquired factor VIII inhibitors, fludarabine-associated immune thrombocytopenic purpura, refractory dermatomyositis, pemphigus vulgaris and myasthenia gravis, and the non-autoimmune conditions of graft versus host disease, acute and chronic transplant rejection, septic shock, insulin resistance, apoptotic conditions, or neoplastic conditions such as multiple myeloma, B-chronic lymphocytic leukaemia and other B cell neoplasias. It should be understood that the subject functioning may correspond to either or both of unwanted immunoglobulin secretion or unwanted antigen presentation. In the context of the latter, therefore, the condition may be characterised by an unwanted T cell response, the efficacy of which T cell response is linked to B cell antigen presentation. Accordingly, by downregulating the level of B cell antigen presentation, for example by down-regulating expansion of the subject B cell population, the efficacy of the unwanted T cell response may be down-regulated.

IDO-mediated tryptophan metabolites or derivatives thereof or pharmaceutically acceptable salts thereof may also be used in conjunction with another therapy, for example chemotherapy or radiotherapy to the extent that a B cell neoplasia is being treated or an immunosuppressive or anti-inflammatory treatment regime to the extent that an autoimmune condition is being treated.

Preferably the present invention is directed to a method for the treatment and/or prophylaxis of an autoimmune condition characterised by aberrant or unwanted B cell functioning in a mammal, said method comprising administering to said mammal an effective amount of one or more IDO-mediated tryptophan metabolites or derivatives thereof or pharmaceutically acceptable salts thereof.

Preferably, the IDO-mediated tryptophan metabolite or derivative thereof is a compound of formula (I), (II) or (III), especially tranilast, 3-HKA, 3-HAA, PA or QA.

Preferably, said condition is rheumatoid arthritis, multiple sclerosis, Crohn's disease, systemic lupus erythematosus, inflammatory bowel disease, type 1 diabetes, psoriasis, acute transplant rejection, chronic transplant rejection, Graves' disease, autoimmune thyroiditis, systemic sclerosis, chronic immune thrombocytopenic purpura, autoimmune haemolytic anaemia, autoimmune polyneuropathy, Wegener's granulomatosis, cold agglutinin disease associated with indolent lymphoma, idiopathic membranous neuropathy, type II mixed cryoglobulinaemia, acquired factor VIII inhibitors, fludarabine-associated immune thrombocytopenic purpura, refractory dermatomyositis, pemphigus vulgaris and myasthenia gravis, GVHD, septic shock, insulin resistance and apoptotic conditions.

More preferably, said B cell functioning is B cell proliferation.

In a related aspect, it has been surprisingly determined that IDO-mediated tryptophan metabolites or derivatives thereof or pharmaceutically acceptable salts thereof, especially compounds of formula (I), are particularly advantageous in the context of the treatment of rheumatoid arthritis. Without limiting the present invention to any one theory or mode of action, in the context of rheumatoid arthritis it is believed that the subject compounds in fact act more broadly, at the cellular level, than just down-regulating B cell functioning, thereby providing an extremely effective means for treating both this particular disorder and all forms of inflammatory joint disease In fact, following treatment with tranilast, subjects exhibited reduced clinical scores, reduced levels of paw-swelling and reduced levels of synovitis, cartilage loss and bone erosion, relative to untreated animals.

Accordingly, a related aspect of the present invention is directed to a method for the treatment and/or prophylaxis of inflammatory joint disease in a mammal, said method comprising administering to said mammal an effective amount of one or more IDO-mediated tryptophan metabolites or derivatives thereof or pharmaceutically acceptable salts thereof.

Preferably, the IDO-mediated tryptophan metabolite or derivative thereof is a compound of formula (I), (II) or (III), especially tranilast, 3-HKA, 3-HAA, PA or QA.

Reference to “inflammatory joint disease” should be understood as a reference to disease conditions which are characterised by the inflammation of tissue which is localised to the skeletal joint regions. This tissue includes the cartilaginous, fibrous and soft (synovial) tissue which lines the opposing surfaces of bone which make up the joint. Reference to “joint” should be understood as a reference to the three classes of joint, being diarthrosis, amphiarthrosis and synarthrosis joints. The subject inflammation may be the result of any cause or aetiology and is not limited to inflammation resulting from the autoimmune condition of rheumatoid arthritis. In a preferred embodiment, however, said inflammatory joint disease is rheumatoid arthritis.

Accordingly, a related aspect of the present invention is directed to a method for the treatment and/or prophylaxis of rheumatoid arthritis in a mammal, said method comprising administering to said mammal an effective amount of one or more IDO-mediated tryptophan metabolites or derivatives thereof or pharmaceutically acceptable salts thereof.

Preferably, the IDO-mediated tryptophan metabolite or derivative thereof is a compound of formula (I), (II) or (III), especially tranilast, 3-HKA, 3-HAA, PA or QA.

An “effective” amount means an amount necessary at least partly to attain the desired response, or to delay the onset or inhibit progression or halt altogether, the onset or progression of a particular condition being treated. The amount varies depending upon the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials.

Reference herein to “treatment” and “prophylaxis” is to be considered in its broadest context. The term “treatment” does not necessarily imply that a subject is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. In the context of rheumatoid arthritis, for example, this may include the amelioration or prevention of inflammation in some joints but not necessarily all joints. This could occur, for example, where the subject compound is administered locally into some but not all affected joints. The term “prophylaxis” may be considered as reducing the severity or onset of a particular condition. “Treatment” may also reduce the severity of an existing condition.

Administration of the IDO-mediated tryptophan metabolites or derivatives thereof or pharmaceutically acceptable salts thereof or antagonists thereof (herein referred to as “modulatory agent”), in the form of a pharmaceutical composition, may be performed by any convenient means. The modulatory agent of the pharmaceutical composition is contemplated to exhibit therapeutic activity when administered in an amount which depends on the particular case. The variation depends, for example, on the human or animal and the modulatory agent chosen. A broad range of doses may be applicable. Considering a patient, for example, from about 0.1 mg to about 1 mg of modulatory agent may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation.

The modulatory agent may be administered in a convenient manner such as by the oral, intravenous (where water soluble), intraperitoneal, intramuscular, subcutaneous, intradermal or suppository routes or implanting (eg. using slow release molecules). The modulatory agent may be administered in the form of pharmaceutically acceptable nontoxic salts, such as acid addition salts or metal complexes, eg. with zinc, iron or the like (which are considered as salts for purposes of this application). Illustrative of such acid addition salts are hydrochloride, hydrobromide, sulphate, phosphate, Maleate, acetate, citrate, benzoate, succinate, maleate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

The modulatory agent may be linked, bound or otherwise associated with any proteinaceous or non-proteinaceous molecules. For example, in one embodiment of the present invention said modulatory agent may be associated with a molecule which permits targeting to a localised region.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip, patch and implant.

In accordance with these methods, the agent defined in accordance with the present invention may be coadministered with one or more other compounds or molecules. By “coadministered” is meant simultaneous administration in the same formulation or in two different formulations via the same or different routes or sequential administration by the same or different routes. For example, the subject agent may be administered together with an agonistic agent in order to enhance its effects. By “sequential” administration is meant a time difference of from seconds, minutes, hours or days between the administration of the two types of molecules. These molecules may be administered in any order.

Yet another aspect of the present invention is directed to the use of one or more IDO-mediated tryptophan metabolites or derivatives thereof or pharmaceutically acceptable salts thereof in the manufacture of a medicament for the treatment of a condition characterised by aberrant or unwanted B cell functioning.

Preferably, said B cell functioning is B cell proliferation or antibody production.

Preferably, said condition is multiple sclerosis, Crohn's disease, systemic lupus erythematosus, inflammatory bowel disease, type 1 diabetes, psoriasis, acute transplant rejection, chronic transplant rejection, Graves' disease, autoimmune thyroiditis, systemic sclerosis, chronic immune thrombocytopenic purpura, autoimmune haemolytic anaemia, autoimmune polyneuropathy, Wegener's granulomatosis, cold agglutinin disease associated with indolent lymphoma, idiopathic membranous neuropathy, type II mixed cryoglobulinaemia, acquired factor VIII inhibitors, fludarabine-associated immune thrombocytopenic purpura, refractory dermatomyositis, pemphigus vulgaris and myasthenia gravis, graft versus host disease, septic shock, insulin resistance and apoptotic conditions.

Still another aspect of the present invention is directed to the use of one or more IDO-mediated tryptophan metabolites or derivatives thereof or pharmaceutically acceptable salts thereof, in the manufacture of a medicament for the treatment of inflammatory joint disease.

Preferably, said inflammatory joint disease is rheumatoid arthritis.

Preferably, the IDO-mediated tryptophan metabolite or derivative thereof is a compound of formula (I), (II) or (III), in particular tranilast, 3-HKA, 3-HAA, PA or QA.

Yet another aspect of the present invention is directed to the use of one or more IDO-mediated tryptophan metabolites or derivatives thereof or pharmaceutically acceptable salts thereof.

Preferably, the IDO-mediated tryptophan metabolite or derivative thereof is a compound of formula (I), (II) or (III), in particular tranilast, 3-HKA, 3-HAA, PA or QA.

The present invention contemplates the administration of the one or more IDO-mediated tryptophan metabolites or derivatives thereof or pharmaceutically acceptable salts thereof either alone or as a pharmaceutical composition comprising one or more IDO-mediated tryptophan metabolites or derivatives thereof or a pharmaceutically acceptable salt thereof or antagonist thereof as hereinbefore defined and one or more pharmaceutically acceptable carriers and/or diluents. Said agents are referred to as the active ingredients.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

Yet another aspect of the present invention relates to IDO-mediated tryptophan metabolites or derivatives thereof or pharmaceutically acceptable salts thereof or antagonists thereof, as hereinbefore defined, when used in the method of the present invention.

The present invention is further defined by the following non-limiting examples.

### Example 1

### Effect of Tranilast on Established Collagen-Induced Arthritis

### Materials and Methods

### Preparation of Type II Collagen

Bovine CII was purified and prepared as previously described (9) and solubilised by stirring overnight at 4° C. in 0.1M acetic acid.

### Immunization of Mice

Male DBA/1 mice (7-8 animals/group) were immunised i.d. at 8-12 weeks of age with bovine CII (200 μg/mouse), emulsified in CFA (Difco Laboratories, West Moseley, UK). Beginning at 14 days after immunisation, mice were inspected daily for signs of arthritis and treatment was initiated on day 1 of arthritis. This research was approved by the local Ethical Review Process Committee and by the Home Office of the United Kingdom.

### Treatment of Arthritis

Tranilast was dissolved in 1% NaHCO₃ by heating to 70° C. and injected i.p. at 100, 200 or 400 mg/kg/day.

### Clinical Assessment of Arthritis

The development of arthritis was assessed daily for the duration of the experiment. The clinical severity of arthritis was graded as follows, 0=normal, 1=slight swelling and/or erythema, 2=pronounced oedematous swelling. Each limb was graded, allowing a maximal clinical score of 8 for each animal. Swelling of hind paws was recorded with a pair of calipers. All clinical evaluations were performed in a blinded manner.

### Histological Assessment of Arthritis

At the end of the treatment period the mice were killed, bled and their joints were processed for histology. The first limb to show clinical evidence of arthritis was removed, fixed, decalcified, and embedded before sectioning and staining with haemotoxylin and eosin. Saggital sections were examined by microscopy in a blinded fashion and joints were graded as follows: 0=normal; 1=mild synovitis without cartilage loss or bone erosion; 2=moderate or severe synovitis with erosions present but normal joint architecture intact; 3=severe synovitis with extensive erosions and normal joint architecture disrupted.

### Results

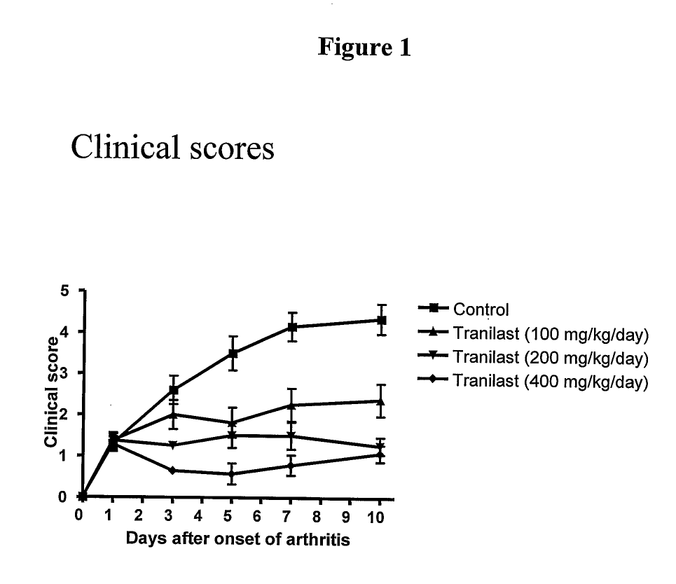

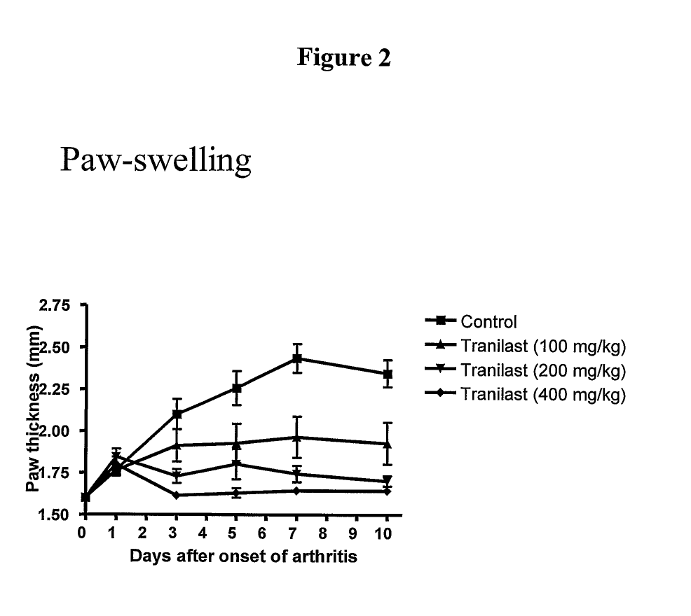

Mice with established collagen-induced arthritis were injected intraperitoneally with tranilast at 100, 200 or 400 mg/kg/day for a period of 10 days. Controls received vehicle alone. During this treatment period clinical scores were assessed and paw swelling was monitored using calipers. It was observed that tranilast reduced clinical scores in a dose-dependent fashion, with 400 mg/kg/day giving maximal suppression of clinical score (FIG. 1). In addition, it was observed that tranilast reduced paw-swelling in a dose-dependent fashion (FIG. 2). At the end of the 10 day treatment period, mice were killed and joints were processed for histology. Histological assessment was performed in a blinded fashion as described in this Examples. There was a reduced level of synovitis, cartilage loss and bone erosion in the mice treated with tranilast, compared to control mice. This was reflected in the significantly reduced histological scores observed in the groups treated with tranilast at 200 or 400 mg/kg (Table 1).

TABLE 1
Tranilast reduces the histological severity of collagen-induced arthritis.

| | | Histological score | |
|---|---|---|---|
| Treatment | N | (mean ± SE) | P value (vs controls) |
| Control | 10 | 2.45 ± 0.24 | |
| Tranilast (100 mg/kg) | 7 | 1.64 ± 0.35 | NS |
| Tranilast (200 mg/kg) | 8 | 1.19 ± 0.35 | P < 0.05 |
| Tranilast (400 mg/kg) | 7 | 0.57 ± 0.20 | P < 0.01 |

DBA/1 mice were immunised with bovine type II collagen in complete Freund's adjuvant in order to induce arthritis. After onset of clinical disease mice were randomly assigned to different treatment groups and given tranilast at 100, 200 or 400 mg/kg/day (i.p.) or vehicle control. Treatment was continued for 10 days after which time the mice were killed and paws were processed for histology. Joints were graded histologically in a blinded fashion.

### Example 2

### Detection of B-Cell Proliferation Using FACS Analysis

### Materials and Methods

### B Cell Purification

All centrifugations were performed at 1500 rpm for 5 min.

B cells were prepared for mouse spleen using rat anti-mouse IgM microbeads and the MACS system.

3 spleens were removed from male DBA/1 mice aged 8-12 weeks. A single cell suspension was prepared by cell-sieve.

Red blood cells were lysed by the addition of 5 ml red blood cell lysis buffer (Sigma) and incubation for 5 min. 5 ml RPMI was added to the cell suspension, and following 2 washes, viable cells were counted with trypan blue (Sigma).

Cells were resuspended in IMAG buffer (BD) at 90 μl/10×10⁶ cells, 10 μl rat anti mouse IgM microbeads (MACS) were added per 10×10⁶ cells.

Cell-bead suspension was incubated in the fridge for 15 min.

Mini MACS columns (1 per 7×10⁷ cells) were placed in a magnet (MACS) and washed with 0.5 ml MACS buffer by gravity flow.

Columns were washed three times with 0.5 ml IMAG buffer.

Columns were removed from the magnet. 2 ml IMAG buffer was added to each column, and B cells were removed pushing through the solution with a plunger.

Cells were cultured at 1×10⁶ cells/ml in 2 ml complete RPMI with glutamine (10% FCS, 1% Pen/Strep, 50 μM 2-Mercaptoethanol, 1 mM Na-Pyruvate) for 48 h in the presence of LPS (20 μg/ml), and 100-25 μg/ml tranilast, or vehicle (DMSO).

20 μM BrdU (Sigma) was added to each well, and incubated overnight.

Cells were then harvested by pipetting, and stained in FACS tubes for either BrdU alone (very sensitive, but does not allow cell-surface staining), or CD40, CD19 and BrdU (less sensitive, but allows co-localisation).

### Single-Staining BrdU Protocol

The pellet was resuspended in 1% formaldehyde in PBS for 5 min at room temperature, and washed in 2 ml PBS.

Ice cold 70% ethanol was added to the pellet (with vortexing), and incubated for a minimum of overnight at −20° C. (generally over the weekend).

Cells were washed twice in 2 ml PBS and pellets resuspended in 0.2 mg pepsin in 1 ml 2N HCl (pH 1.5) (with vortexing) at room temperature for 1 h. Cells were mixed by gentle vortexing occasionally.

1 ml 0.1 M Borax was added to the cell pellet, and following centrifugation cells were washed twice in 2 ml PBS.

The pellet was resuspended in 2 ml PBS (by vortexing) and incubated for 15 min at room temperature, to allow all the pepsin/borax to leach from the cells.

Cells were washed in 2 ml PBS, 1% FCS, 0.1% sodium azide.

100 μl mouse anti-BrdU-FITC with DNAse (BD) antibody diluted 1:10 in PBS, 1% FCS, 0.1% sodium azide was added to the cell pellet and incubated for 30 min at room temperature in the dark.

4 ml PBS was added to the cells to wash.

The cell pellet was resuspended in 100 μl 1% formaldehyde-PBS.

FACS analysis of staining was performed on the BD LSR Flow Cytometer.

### CD40, CD19 and BrdU Staining Protocol

Cells were washed twice in 2 ml PBS, and resuspended in 100 μl PBS/1% FCS, 0.1% sodium Azide, with 2.5 μl anti CD40-RPE (ImmunoKontact) and 5 μl anti CD19-PerCP-CY5.5 (BD). Cells were incubated for 30 min at room temperature in the dark.

4 ml PBS was added to the cells to wash, and pellet was resuspended in 0.5 ml ice-cold 0.15M NaCl.

1.2 ml ice cold 95% ethanol was added to the tube with gentle vortexing, and cells were incubated for 30 min on ice.

2 ml PBS was added to the tube, to wash cells, and the pellet was resuspended in 1 ml 1% formaldehyde-PBS. Cells were incubated for 30 min at room temperature.

Cells were pelleted, and resuspended in 100 μl PBS, 1% BSA, 0.1% sodium azide containing 10 μl anti-BrdU DNAse (BD), and incubated for 30 min at room temperature in the dark.

Cells were washed with the addition of 4 ml PBS, and resuspended in 100 μl 1% formaldehyde-PBS.

FACS analysis of staining was performed on the BD LSR Flow Cytometer.

### CD40 Stimulation

For CD40 stimulation, the cells were grown as for LPS stimulation (in 200 μl in 96 well plates) with 20 μg azide-free anti-mouse CD40.

### Results

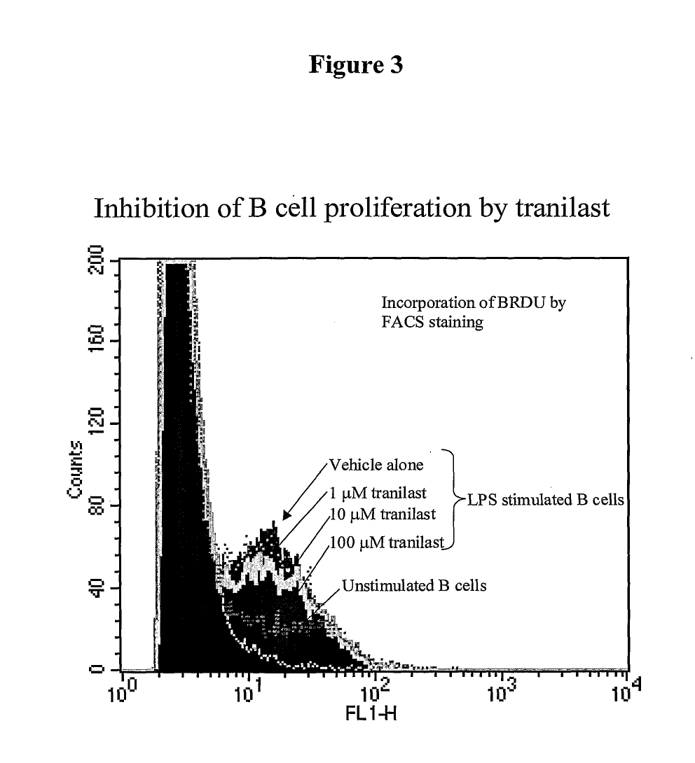

A study was performed to assess the influence of tranilast on B cell proliferation. B cells were cultured in vitro with LPS and 1 μg-100 μg tranilast. Cells were cultured with BrdU, and uptake measured by FACs analysis. LPS induced proliferation (BrdU labelling) in 75% of B cells. Tranilast inhibited the proliferation dose-dependently (FIG. 3). The maximum inhibition of proliferation observed was 75%, with 100 μg/ml tranilast.

### Example 3

### [³H] Detection of B Cell Proliferation

### Materials and Methods

### B Cell Purification and Stimulation—as for FACS

All centrifugations were performed at 1500 rpm for 5 min.

B cells were prepared from mouse spleen using rat anti-mouse IgM microbeads and the MACS system.

3 spleens were removed from male DBA/1 mice aged 8-12 weeks. A single cell suspension was prepared by cell-sieve.

Red blood cells were lysed by the addition of 5 ml red blood cell lysis buffer (Sigma) and incubation for 5 min. 5 ml RPMI was added to the cell suspension, and following 2 washes, viable cells were counted with trypan blue (Sigma).

Cells were resuspended in IMAG buffer (BD) at 90 μl/10×10⁶ cells. 101 rat anti-mouse IgM microbeads (MACS) were added per 10×10⁶ cells.

Cell-bead suspension was incubated in the fridge for 15 min.

Mini MACS columns (1 per˜7×10⁷ cells) were placed in a magnet (MACS) and washed with 0.5 ml MACS buffer by gravity flow.

Columns were washed three times with 0.5 ml IMAG buffer.

Columns were removed from the magnet. 2 ml IMAG buffer was added to each column, and B cells were removed pushing through the solution with a plunger.

### Tritium-Pulsing of Cells and Harvesting

Cells were cultured at 1×10⁶ cells/ml in 200 μl complete RPMI with glutamine (10% FCS, 1% Pen/Strep, 50 μM 2-Mercaptoethanol, 1 mM Na-Pyruvate) in a 96-well plate for 48 h in the presence of LPS (20 μg/ml), azide-free anti-CD40 monoclonal antibody (10 μg/ml), or azide free F(ab′)₂ fragment of anti-IgM monoclonal antibody (20 μg/ml) and 100-6.25 μg/ml tranilast, or vehicle (DMSO).

1 μCi ³[H]thymidine was added to each well and incubated overnight. Cells were harvested onto a pre-wet filter mat using a Skaton cell harvester. The filter mat was dried in a microwave for 2 minutes at 750 W. The dry filter mat was sealed in a bag using a plate sealer, and the corner cut. 10 ml scintillation fluid was placed in the bag, and spread evenly over the filter mat. The bag was re-sealed and read in a Wallac 1205 plate reader.

### Results

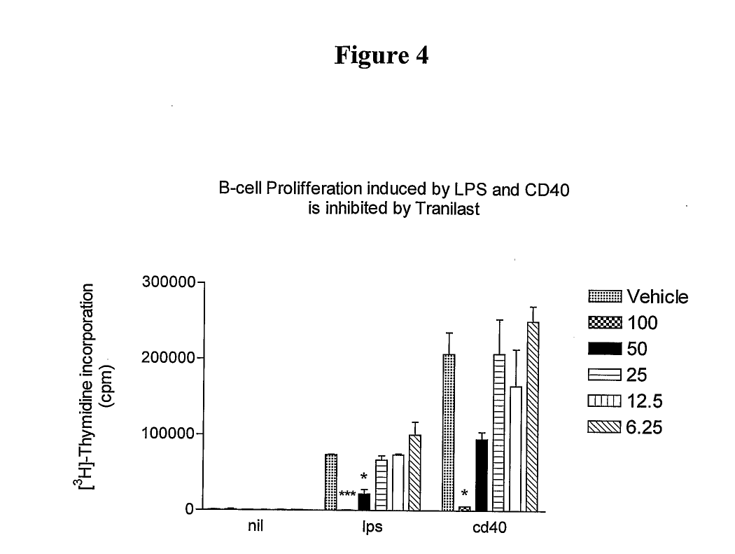

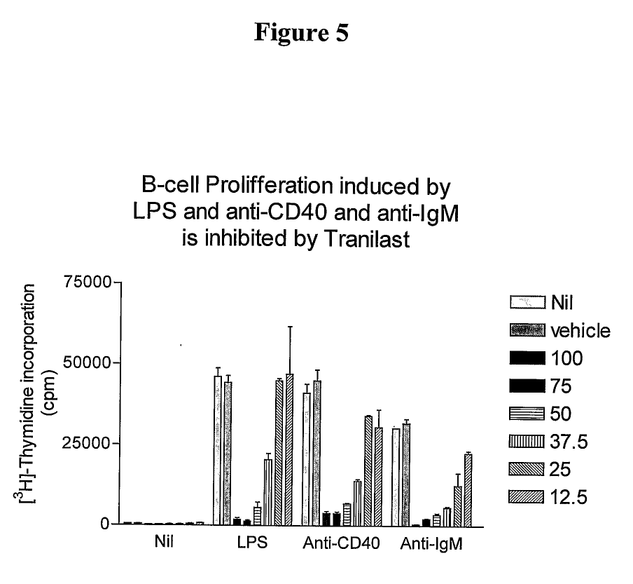

The influence of tranilast on B cell proliferation in vitro was assessed using ³[H]thymidine incorporation. B cells were activated with either LPS, anti-CD40, or anti-IgM antibodies, and given up to 100 μg/ml tranilast. Cells were cultured with ³[H]thymidine, and uptake assessed. LPS induced a 92- (FIG. 4) and 91-fold (FIG. 5) increase in thymidine uptake in B-cells, which was inhibited dose-dependently by tranilast. A maximum inhibition of 99% was observed with 100 μg/ml tranilast. Anti-CD40 antibody induced a 238- (FIG. 4) and 81-fold (FIG. 5) increase in thymidine uptake. A dose-dependent inhibition was again observed with tranilast treatment, with a maximal 97% inhibition of proliferation detected with 100 μg/ml tranilast. Anti-IgM antibody induced a 60-fold increase (FIG. 5) in B cells proliferation. Tranilast inhibited anti-IgM induced proliferation dose-dependently and was effective at all doses assessed.

### Example 4

### Effect of Tranilast and 3-Hydroxyanthranilic Acid on Established Collagen-Induced Arthritis

### Materials and Methods

### Reagents

Type II collagen was purified from bovine cartilage, as described [Williams, R. O. 2004, Methods Mol. Med. 98:207-216] and solubilized by stirring overnight at 4° C. in acetic acid (0.1M) or Tris buffer (0.05 M Tris, containing 0.2 M NaCl, pH 7.4). Tranilast was synthesised by Angiogen Pharmaceuticals Pty. Ltd. For in vivo studies Tranilast was dissolved at a maximum concentration of 10 mg/ml in 1% sodium bicarbonate by heating for 1 h at 70° C. Upon cooling, an emulsion was formed. For in vitro studies Tranilast was dissolved in dimethyl sulphoxide (DMSO). 3-Hydroxy-anthranilic acid (3-HAA) was purchased from Sigma (Poole, UK) and dissolved in PBS.

### Induction and Assessment of Arthritis

Male DBA/1 mice (8-12 weeks old) were immunized intradermally at the base of the tail with bovine type II collagen (200 μg) emulsified in complete Freund's adjuvant (CFA; Difco, West Moseley, UK). Arthritis was monitored clinically using the following scoring system: 0=normal, 1=slight swelling and/or erythema, and 2=pronounced oedematous swelling. Each limb was graded, giving a maximum score of 8 per mouse. In addition, paw-swelling was measured using calipers.

Histopathological assessment of arthritis was carried out in a blinded fashion on decalcified haematoxylin and eosin stained sections using a scoring system as follows: 0, normal; 1, minimal synovitis without cartilage/bone erosion; 2, synovitis with some marginal erosion but joint architecture maintained; 3, severe synovitis and erosion with loss of normal joint architecture. This research was approved by the local ethical review process committee and by the Home Office of Great Britain.

### Serum Anti-Collagen Antibody Levels

ELISA plates (Nunc, Uxbridge, UK) were coated with 2 μg/ml of bovine CII dissolved overnight in Tris Buffer (0.05 M Tris, containing 0.2 M NaCl, pH 7.4) blocked with 2% bovine serum albumin (BSA) and then incubated with serial dilutions of test sera. A reference sample was included on each plate. Bound total IgG, IgG1 or IgG2a was detected by incubation with HRP-conjugated sheep anti-mouse IgG, IgG1 or IgG2a, followed by TMB substrate. Optical density was measured at 450 nm.

### Analysis of T Cell Responses

Inguinal lymph nodes were excised from Tranilast-treated and control mice. Alternatively, inguinal lymph nodes were removed from untreated arthritic mice (day 1-5 of arthritis) and Tranilast was added in vitro. In both cases, LNC were cultured in RPMI 1640 containing FCS (10% v/v), 2-mercaptoethanol (20 μM), L-glutamine (1% w/v), penicillin (100 U/ml) and streptomycin (100 μg/ml) in the presence or absence of type II collagen (50 μg/ml). Secreted cytokines (IFN-γ, IL-5, TNFα and IL-10) were measured after 72 h. by ELISA. In brief, 96 well ELISA plates were coated with the respective capture antibody, blocked with bovine serum albumin (2% w/v), and then incubated with LNC culture supernatants (neat) overnight at 4° C. After washing, bound cytokines were detected using biotinylated detect antibodies. A standard curve was generated using known concentrations of the appropriate recombinant cytokine and the concentrations of cytokines present in culture supernatants were estimated by reference to the standard curve.

B and T cell Purification and Proliferation

A single cell suspension was prepared from spleen by mincing through a cell strainer, and erythrocytes were lysed using an ammonium chloride solution (Sigma, St Louis, Mo., USA). B cells were positively enriched by using anti-IgM MACS microbeads, and T cells were positively enriched using anti-CD4 MACS microbeads and the MACS system, according to the manufacturer's guidelines (Miltenyi Biotec, Bergisch Gladbach, Germany). Purity was assessed by flow cytometric analysis (B cell>90% CD19+, T cell>90% CD4+). Cells were cultured at 5×10⁵ cells/ml in 200 μl complete RPMI, as above, in a flat bottom 96-well plate and cultured for 72 h. B cells were stimulated with anti-CD40 monoclonal antibody (10 μg/ml; BD), and T cells were stimulated with 5 μg/ml plate-bound anti-CD3 (BD), 5 μg/ml soluble anti-CD28 (BD). Tranilast, 3-HAA, or vehicle (DMSO) were incubated at concentrations stated with the cultures. 48 hours after stimulation, 100 μl culture medium was collected, and cells were pulsed with 1 μCi ³H thymidine per well for 18 h. Cells were then harvested and plates assessed for incorporation. Each assay was performed on a minimum of 3 occasions. Data shown is of one representative experiment, and is expressed as mean+/−SD of triplicate in culture. IFN-γ, IL-10 and IL-5 levels was assessed in the culture medium by ELISA, as above.

### Statistical Analysis

Group means were analysed by one-way analysis of variance, followed by the Dunnett Multiple Comparisons test, where appropriate.

### Results

### Tranilast Inhibits Development of Collagen-Induced Arthritis

In order to assess its anti-arthritic potential, Tranilast was injected into DBA/1 mice (200 mg/kg/day) from the day of immunisation with type II collagen in CFA. By day 28, 5 of 7 (71%) vehicle treated mice had developed arthritis of moderate severity (clinical score 2.8±0.6), whilst 1 of 7 (14%) Tranilast-treated mice had developed mild arthritis (clinical score 1). Analysis of the sera of treated and control mice revealed no change in anti-collagen IgG1 or IgG2a levels in Tranilast-treated mice.

### Tranilast Reduces the Severity of Established Arthritis

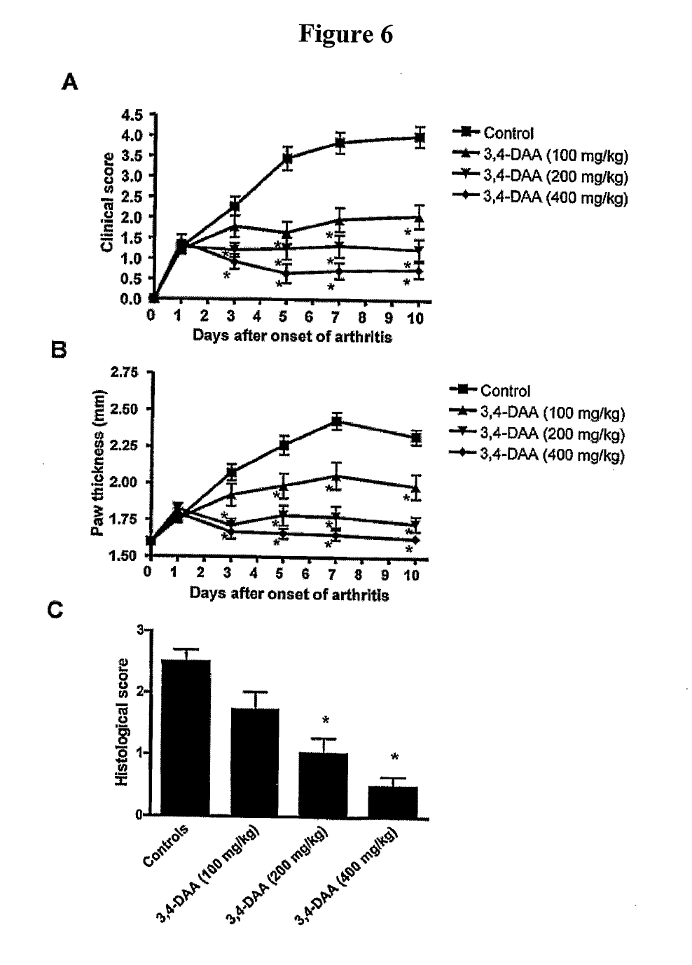

The ability of Tranilast to treat established CIA was tested. Mice were immunised with type II collagen in CFA. On day 1 of clinical arthritis (the day that arthritis was first observed) mice were randomly assigned to different treatment groups and given Tranilast (100 mg/kg/day, 200 mg/kg/day or 400 mg/kg/day) or vehicle alone over a 10 day period. In two separate experiments, a dose-dependent reduction in both clinical scores and paw-swelling was observed in the Tranilast-treated mice (FIG. 6). Significant differences between Tranilast treated and control mice were observed from day 3 until the end of the treatment period (day 10). On day 10 the mice were killed and the first paw to show clinical evidence of arthritis was processed for histology. Joints were examined blindly for severity of inflammation and joint erosion. Again, a clear dose-dependent reduction in histological severity of arthritis was observed in Tranilast-treated mice (FIG. 6).

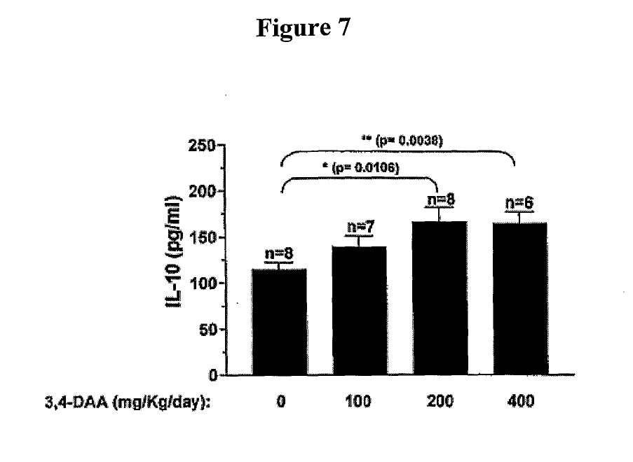

Sera from control and treated mice were analysed for levels of anti-type II collagen IgG1 and IgG2a but no differences were observed between any of the groups. Sera were also analysed for IL-10 production and a dose-dependent increase in circulating IL-10 levels was detected following treatment with Tranilast (FIG. 7).

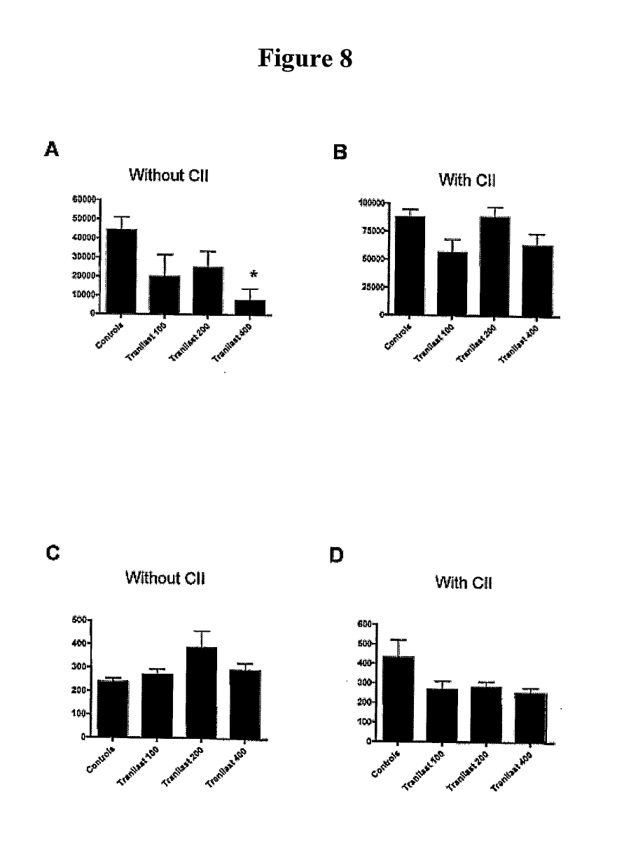

At the end of the experiment draining (inguinal) LNC from control and treated mice were cultured for 72 h in the presence or absence of type II collagen. IFN-γ and IL-5 production was measured by ELISA. IFNγ production was found to be significantly reduced in the mice given Tranilast at 400 mg/mouse (FIG. 8). However, on restimulation with collagen, differences between the groups were not significant, indicating that the ability of the T cells to respond to antigenic stimulation returned to normal once the Tranilast had been removed from the system. IL-5 production was unaffected by treatment with Tranilast.

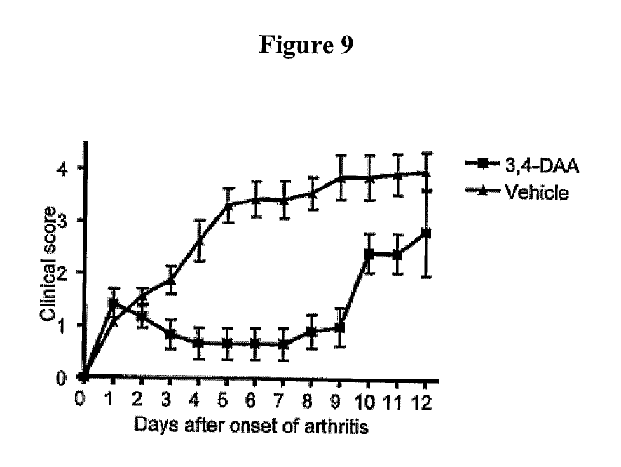

It is clearly of interest to establish what happens when treatment with Tranilast is stopped. Is there a disease flare and if so, does it occur immediately after cessation of treatment? Hence, a group of arthritic mice were treated from day 1 to day 5 of arthritis with Tranilast (400 mg/kg/day) (FIG. 9). Treatment was then stopped and mice were monitored for a further 7 days. As before, there was a dramatic reduction in arthritis severity during the treatment period. When treatment was stopped on day 5, exacerbation of arthritis was observed from day 9, although the severity of arthritis did not reach that of the control group.

### Tranilast Inhibits B and T Cell Proliferation In Vitro

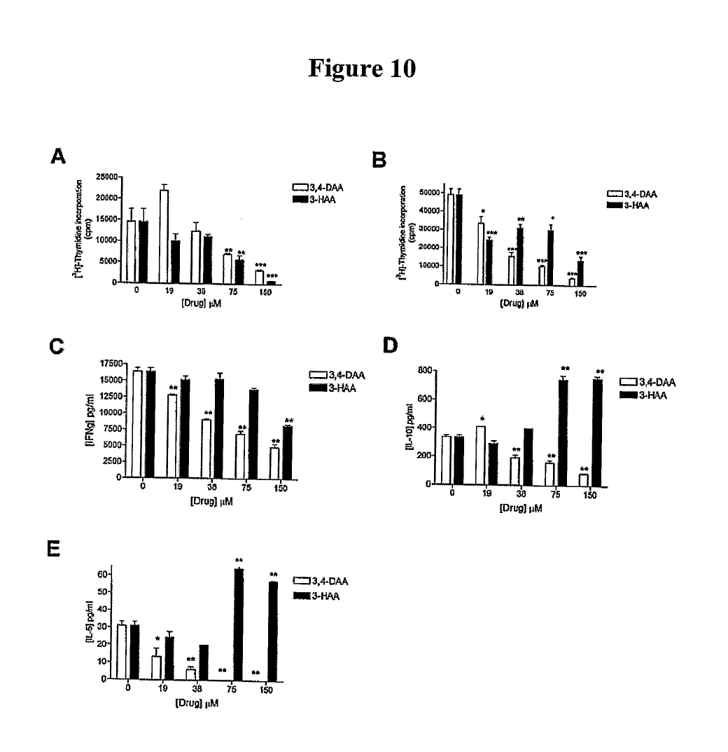

To investigate potential mechanisms of anti-inflammatory actions of Tranilast, anti-proliferative action of Tranilast at therapeutic concentrations was compared with its natural analogue, 3-HAA against both B and T cells (FIG. 10). Activation of purified B (FIG. 10A) and T (FIG. 10B) cells was induced by anti-CD40, and anti-CD3/CD28 respectively, and proliferation was assessed by ³H-thymidine incorporation. Both Tranilast and 3-HAA dose-dependently inhibited B and T cell proliferation. The IC₅₀ was calculated for each drug. The IC₅₀ of Tranilast and 3-HAA for inhibition of B cell proliferation was 73.09 μM and 64.66 μM respectively. However, the IC₅₀ for inhibition of T cell proliferation was 27.99 μM for Tranilast, and 100.12 μM for 3-HAA. Both Tranilast and 3-HAA therapy dose-dependently reduced IFN-γ production by T-cells (FIG. 10C). In contrast Tranilast dose-dependently inhibited IL-10 production (FIG. 10D), whilst 3-HAA increased IL-10 production by T-cells, indicating Tranilast may act via additional mechanisms to 3-HAA.

### Example 5

### Inhibition of Anti-Type II Collagen Antibody Production by Tranilast

Earlier examples show that tranilast has a potent anti-proliferative effect on B cells stimulated in vitro with lipopolysaccharide, anti-CD40 mAb or anti-IgM antibody. The question was therefore addressed as to whether tranilast would inhibit antibody responses in vivo and would therefore be useful in the treatment of autoimmune diseases in which antibodies play a pathogenic role, including rheumatoid arthritis, Grave's disease, Hashimoto's thyroiditis, Sjogren's syndrome and systemic lupus erythematosus.

### Materials and Methods

Female DBA/1 mice (10 weeks of age) were immunised intraperitoneally on day 1 with 100 μg bovine type II collagen, dissolved in 0.05M Tris/0.2M NaCl, pH 7.4). Tranilast was dissolved in 1% sodium bicarbonate (10 mg/ml) by heating for 1 h at 70° C. Upon cooling, an emulsion was formed. Tranilast was administered intraperitoneally for three weeks (starting on day 1) at a dose of 400 mg/kg, every 2-3 days. Controls received vehicle alone. There were six mice per group.

On day 28 mice were bled and serum levels of anti-type II collagen IgG1 and IgG2a were measured by ELISA, as follows:

1. Polystyrene microtitre plates (Immulon 2, Dynatech Laboratories) were coated with DEAE-purified bovine type II collagen, dissolved in 0.2M NaCl/0.05M Tris, pH 7.4 (5 μg/ml), overnight at 4° C.
2. The plates were washed in phosphate-buffered saline (PBS), then blocked with BSA in PBS (2% w/v) for 1 h at room temperature.
3. After washing in PBS, containing Tween 20 (0.05% v/v), the sera were serially diluted in PBS/Tween, added to the plates and incubated for 2 h at room temperature.
4. After washing in PBS/Tween, bound IgG was detected by incubating goat anti-mouse IgG1 or anti-mouse IgG2a-HRP conjugate ( 1/1000) for 2 h at room temperature.
5. Develop colour reaction using TMB substrate. Stop reaction with 4.5NH₂SO₄
6. The plates were read at 450 nm on a microtitre plate reader.

### Results

One mouse from the control group died during the study and could not be bled. Levels of anti-type II collagen IgG1 were significantly higher in the control group versus the Tranilast treated group (P=0.03; Mann Whitney test). Collagen-specific IgG2a antibodies were not detected. Optical densities and endpoint titres for individual mice are shown in Table 2.

TABLE 2
Levels of anti-type II collagen IgG1 in mice
treated with tranilast versus controls.

| Mouse | Treatment | OD₄₅₀ (dilution 1/100) | Titre (reciprocal) |
|---|---|---|---|
| 1 | Control | 0.21 | 100 |
| 2 | Control | 0.12 | 100 |
| 3 | Control | 0.14 | 50 |
| 4 | Control | 0.95 | 400 |
| 5 | Control | 0.66 | 400 |
| 6 | Tranilast | 0.07 | <50 |
| 7 | Tranilast | 0.06 | <1/50 |
| 8 | Tranilast | 0.31 | 150 |
| 9 | Tranilast | 0.08 | <50 |
| 10 | Tranilast | 0.06 | <50 |
| 11 | Tranilast | 0.08 | <50 |

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Ahmad et al., Am. J. Hematol. 2004; 77(2): 171-176
Akashi T., Nagafuchi S., Anzai K., Kondo S., Kitamura D., Wakana S., Ono J., Kikuchi M., Niho Y., Watanabe T. Direct evidence for the contribution of B cells to the progress of insulitis and the development of diabetes in non-obese diabetic mice. Int. Immunol. 1997; 9(8): 1159-1164.
Alter A., Duddy M., Herbert S., Biernacki K., Prat A., Antel J. P., Yong V. W., Nuttall R. K., Pennington C. J., Edwards D. R., Bar-Or A. Determinants of human B cell migration across brain endothelial cells. J. Immunol. 2003; 170: 4497-4505.
Anolik J., Sanz I. B cells in human and murine systemic lupus erythematosus. Curr. Opin. Rheumatol. 2004; 16(5): 505-512.
Archelos J. J., Storch M. K., Hartung H. P. The role of B cells and autoantibodies in multiple sclerosis. Ann. Neurol. 2000; 47(6): 694-706.
Asano et al., Am. J. Pathol. 2004; 165(2): 641-650
Avivi et al., Br. J. Cancer. 2003; 89(8): 1389-1394
Bachelez H., Flageul B., Dubertret L., Fraitag S., Grossman R., Brousse N., Poisson D., Knowles R. W., Wacholtz M. C., Haverty T. P., Chatenoud L., Bach J. F. Treatment of recalcitrant plaque psoriasis with a humanized non-depleting antibody to CD4. J. Autoimmun. 1998; 11(1): 53-62.
Baker K. P. BLyS—an essential survival factor for B cells: basic biology, links to pathology and therapeutic targets. Autoimmun. Rev. 2004; 3(5): 368-375.
Becker Y. T., Becker B. N., Pirsch J. D., Sollinger H. W. Rituximab as a treatment for refractory kidney transplant. Am. J. Transplant 2004; 4(6): 996-1001.
Berentsen et al., Blood 2004; 103(8): 2925-2928
Boye et al., Ann. Oncol. 2003; 14(4): 520-535
Boyman O., Hefti H. P., Conrad C., Nickoloff B. J., Suter M., Nestle F. O. Spontaneous development of psoriasis in a new animal model shows an essential role for resident T cells and tumor necrosis factor-α. J. Exp. Med. 2004; 199(5): 731-736.
Burgoon M. P., Gilden D. H., Owens G. P. B cells in multiple sclerosis. Front. Biosci. 2004; 1(9): 786-796.
Chan O. T., Madaio M. P., Shlomchik M. J. The central and multiple roles of B cells in lupus pathogenesis. Immunol. Rev. 1999; 169: 107-121.
Chan O. T. M., Hannum L. G., Haberman A. M., Madaio M. P., Shlomchik M. J. A novel mouse with B cells but lacking serum antibody reveals an antibody-independent role for B cells in murine lupus. J. Exp. Med. 1999; 189(10): 1639-1648.
Chan O. T. M., Madaio M. P., Shlomchik M. J. B cells are required for lupus nephritis in the polygenic, Fas-intact MRL model of systemic autoimmunity. J. Immunol. 1999; 163(7): 3592-3596.
Chiu P. P., Serreze D. V., Danska J. S. Development and function of diabetogenic T-cells in B-cell deficient nonobese diabetic mice. Diabetes 2001; 50(4): 763-770.
Cohen et al., Leuk Lymphoma 2001; 42(6): 1405-1408
Cooper et al., Br. J. Haematol. 2004; 125(2): 232-239
Desai-Mehta A., Lu L., Ramsey-Goldman R., Datta S. K. Hyperexpression of CD40 ligand by B and T cells in human lupus and its role in pathogenic autoantibody production. J. Clin. Invest. 1996; 97(9): 2063-2073.
Dupuy et al., Arch. Dermatol. 2004; 140(1): 91-96
Edwards J. C., Szczepanski L., Szechinski J., Filipowicz-Sosnowska A., Emery P., Close D. R., Shaw T. Efficacy of B-cell-targeted therapy with rituximab in patients with rheumatoid arthritis. N. Engl. J. Med. 2004; 350(25): 2572-2581.
Gajra et al., Am. J. Hematol 2004; 77(2): 196-197
Gerhard et al., Z. Rheumatol. 2002, 61(6):718-727
Gorman C., Leandro M., Isenberg D. B cell depletion in autoimmune disease. Arthritis Res. Ther. 2003; 5 (Suppl 4): S17-S21.
Greeley S. A., Moore D. J., Noorchashm H., Noto L. E., Rostami S. Y., Schlachterman A., Song H. K., Koeberlein B., Barker C. F., Naji A. J. Impaired activation of islet-reactive CD4 T cells in pancreatic lymph nodes of B cell-deficient nonobese diabetic mice. J. Immunol. 2001; 167(8): 4351-4357.
Griffiths C. E. The immunological basis of psoriasis. J. Eur. Acad. Dermatol. Venerol. 2003; 17(Suppl 2): 1-5.
Hasselbalch, Immun. Lett. 2003; 88(1): 85-86
Haubold et al., Ann. Neurol. 2004, 56(1):97-107
Hayer et al., Bone Miner. Res. 2004, 19(6):990-998
Hegde et al., Blood 2002; 100(6): 2260-2262
Hemmer B., Archelos J. J., Hartung H. P. New concepts in the immunopathogenesis of multiple sclerosis. Nat. Rev. Neurosci. 2002; 3(4): 291-301.
Hemmer B., Cepok S., Nessler S., Sommer N. Pathogenesis of multiple sclerosis: an update on immunology. Curr. Opin. Neurol. 2002; 15(3): 227-231.
Hibi T., Aiso S., Ishikawa M., Watanabe M., Yoshida T., Kobayashi K., Asakura H., Tsuru S., Tsuchiya M. Circulating antibodies to the surface antigens on colonic epithelial cells in ulcerative colitis. Clin. Exp. Immunol. 1983; 54(1): 163-168.
Higuchi T., Aiba Y., Nomura T., Matsuda J., Mochida K., Suzuki M., Kikutani H., Honjo T., Nishioka K., Tsubata T. Cutting edge: Ectopic expression of CD40 ligand on B cells induces lupus-like autoimmune disease. J. Immunol. 2002; 168(1): 9-12.
Iglesias A., Bauer J., Litzenburger T., Schubart A., Linington C. T- and B-cell responses to myelin oligodendrocyte glycoprotein experimental autoimmune encephalomyelitis and multiple sclerosis. Glia 2001; 36(2): 220-234.
Ishikawa O., Miyachi Y., Fujita K., Takenoshita S., Nagamachi Y., Ulcerative colitis associated with preceding lupus erythematosus. J. Dermatol. 1995; 22(4): 289-291.
Kawamura T., Kanai T., Dohi T., Uraushihara K., Totsuka T., Iiyama R., Taneda C., Yamazaki M., Nakamura T., Higuchi T., Aiba Y., Tsubata T., Watanabe M. J. Immunol. 2004; 172(10): 6388-6397.
Kendall P. L., Woodward E. J., Hulbert C., Thomas J. W. Peritoneal B cells govern the outcome of diabetes in non-obese diabetic mice. Eur. J. Immunol. 2004; 34(9): 2387-2
Kimball J. A., Pescovitz M. D., Book B. K., Norman D. J. Reduced human IgG anti-ATGAM antibody formation in renal transplant recipients receiving mycophenolate mofetil. Transplantation 1995; 60(12): 1379-1383.
Kritikos H., Mouzas I. A., Spanoudakis S. M., Kapsoritakis A. N., Petinaki E., Kouroumalis E. A., Manousos O. N. Association between ulcerative colitis and systemic lupus erythematosus: report of two cases. Eur. J. Gastroenterol. Hepatol. 1998; 10(5): 437-439.
Krukemeyer et al., Transplantation 2004; 78(1): 65-70
Levine and Pestronk, Neurology 1999; 52(8): 1701-1704
Levine, Arthritis Rheum. 2002; 46 Suppl. 9
Looney R. J., Anolik J., Sanz I. B cells as therapeutic targets for rheumatic diseases. Curr. Opin. Rheumatol. 2004; 16: 180-185.
Looney R. J., Anolik J., Sanz I. B lymphocytes in systemic lupus erythematosus: lessons from therapy targeting B cells. Lupus 2004; 13(5): 381-390.
Looney R. J., Anolik J. H., Campbell D., Felgar R. E., Young F., Arend L. J., Sloand J. A., Rosenblatt J., Sanz I. B cell depletion as a novel treatment for systemic lupus erythematosus: a phase I/II dose-escalation trial of rituximab. Arthritis. Rheum. 2004; 50(8): 2580-2589.
Morel P., Revillard J. P., Nicolas J. F., Wijdenes J., Rizova H., Thivolet J. Anti-CD4 monoclonal antibody therapy in severe psoriasis. J. Autoimmun. 1992; 5(4): 465-477.
Nielsen et al., Eur. J. Immunol., 2004; 34(1): 263-272
Noorchashm H., Lieu Y. K., Noorchashm N., Rostami S. Y., Greeley S. A., Schlachterman A., Song H. K., Jevnikar A. M., Barker C. F., Naji A. I-Ag7-mediated antigen presentation by B lymphocytes is critical in overcoming a checkpoint in T cell tolerance to islet beta cells of nonobese diabetic mice. J. Immunol. 1999; 163(2): 743-750.
Noorchashm H., Noorchashm N., Kern J., Rostami S. Y., Barker C. F., Naji A. B-cells are required for the initiation of insulitis and sialitis in nonobese diabetic mice. Diabetes 1997; 46(6): 941-946.
Oligino T. J., Dalrymple S. A. Targeting B cells for the treatment of rheumatoid arthritis. Arthritis Res. Ther. 2003; 5(Suppl 4): S7-S11.
Olson T. S., Bamias G., Naganuma M., Rivera-Nieves J., Burcin T. L., Ross W., Morris M. A., Pizarro T. T., Ernst P. B., Cominelli F., Ley K. Expanded B cell population blocks regulatory T cells and exacerbates ileitis in a murine model of Crohn disease. J. Clin. Invest. 2004; 114(3): 389-398.
Pescovitz M. D. The use of rituximab, anti-CD20 monoclonal antibody, in pediatric transplantation. Pediatr. Transplant 2004; 8(1): 9-21.
Pescovitz M. D., Book B. K., Henson S., Leapman S. B., Milgrom M. L., Kim J., Norman D., Filo R. S. The addition of sirolimus to cyclosporine and steroids inhibits anti-equine antibody response in renal transplant recipients treated with antithymocyte globulin. Am. J. Transplant. 2003; 3(4): 497-500.
Rastetter et al., Annu. Rev. Med. 2004; 55: 477-503
Qin Y., Duquette P. B-cell immunity in MS. Int. MS J. 2003; 10(4): 110-120.
Qin Y., Duquette P., Zhang Y., Olek M., Da R. R., Richardson J., Antel J. P., Talbot P., Cashman N. R., Tourtellotte W. W., Wekerle H., Van Den Noort S. Intrathecal B-cell clonal expansion, an early sign of humoral immunity, in the cerebrospinal fluid of patients with clinically isolated syndrome suggestive of multiple sclerosis. Lab. Invest. 2003; 83(7): 1081-1088.
Ratanatharathorn et al., Biol. Blood Marrow Transplant 2003; 9(8): 505-511
Ruggenenti et al., J. Am. Soc. Nephrol. 2003; 14(7): 1851-1857
Sadlack B., Merz H., Schorle H., Schimpl A., Feller A. C., Horak I. Ulcerative colitis-like disease in mice with a disrupted interleukin-2 gene. Cell 1993; 75(2): 253-261.
Sarwal M., Chua M.-S., Kambham N., Hsieh S.-C., Satterwhite T., Masek M., Salvatierra O. Molecular heterogeneity in acute renal allograft rejection identified by DNA microarray profiling. N. Engl. J. Med. 2003; 349(2): 125-138.
Sato et al., Arthritis Rheum. 2004; 50(6): 1918-1927
Serreze D. V., Chapman H. D., Varnum D. S., Hanson M. S., Reifsnyder P. C., Richard S. D., Fleming S. A., Leiter E. H., Shultz L. D. B lymphocytes are essential for the initiation of T cell-mediated autoimmune diabetes: analysis of a new “speed congenic” stock of Igμ^{null} mice. J. Exp. Med. 1996; 184(5): 2049-2053.
Serreze D. V., Fleming S. A., Chapman H. D., Richard S. D., Leiter E. H., Tisch R. M. J. Immunol. 1998; 161(8): 3912-3918.
Serreze D. V., Silveira P. A. The role of B lymphocytes as key antigen-presenting cells in the development of T cell-mediated autoimmune type 1 diabetes. Curr. Dir. Autoimmun. 2003; 6: 212-227.
Silveira P. A., Dombrowsky J., Johnson E., Chapman H. D., Nemazee D., Serreze D. V. B cell selection defects underlie the development of diabetogenic APCs in nonobese diabetic mice. J. Immunol. 2004; 172(8): 5086-5094.
Silveira P. A., Johnson E., Chapman H. D., Bui T., Tisch R. M., Serreze D. V. The preferential ability of B lymphocytes to act as diabetogenic APC in NOD mice depends on expression of self-antigen-specific immunoglobulin receptors. Eur. J. Immunol. 2002; 32(12): 3657-3666.
Silverman G. J., Carson D. A. Roles of B cells in rheumatoid arthritis. Arthritis Res. Ther. 2003; 5(Suppl 4): S1-S6.
Silverman G. J., Weisman S. Rituximab therapy and autoimmune disorders. Prospects for anti-B cell therapy. Arthritis Rheum. 2003; 48(6): 1484-1492.
Specks et al., Arthritis Rheum. 2001; 44(12): 2836-2840
Stasi et al., Blood 2001; 98(4): 952-957
Stasi et al., Blood 2004; 103(12): 4424-4428
Svensson L., Abdul-Majid K.-B., Bauer J., Lassmann H., Harris R. A., Holmdahl R. A comparative analysis of B cell-mediated myelin oligodendrocyte glycoprotein-experimental autoimmune encephalomyelitis pathogenesis in B cell-deficient mice reveals an effect on demyelination. Eur. J. Immunol. 2002; 32(7): 1939-1946.
Takahashi F., Das K. M. Isolation and characterization of a colonic autoantigen specifically recognized by colon tissue-bound immunoglobulin from idiopathic ulcerative colitis. J. Clin. Invest. 1985; 76(1): 311-318.
Takemura S., Klimiul P. A., Braun A. Goronzy J. J., Weyand C. M. T cell activation in rheumatoid synovium is B cell dependent. J. Immunol. 2001; 167(8): 4710-4718.
Wiestner et al. Blood 2002; 100(9): 3426-3428
Williams, R. O. 2004. Collagen-induced arthritis as a model for rheumatoid arthritis. Methods Mol. Med. 98:207-216
Wylam et al., J. Pediatr. 2003; 143 (5): 674-677
Zaja et al. Blood 2003; 101(10): 3827-3834
Zaja et al., Neurology 2000; 55(7): 1062-1063
Zampini P., Riviera A. P., Tridente G. In vitro inhibition of histamine release from mouse mast cells and human basophils by an anthranilic acid derivative. Int. J. Immunopharmacol., 1983; 5(5):431-5.
Zecca et al., Blood 2001; 97(12): 3995-97

1. A method of downregulating B cell functioning, said method comprising contacting said B cell with an effective amount of one or more IDO-mediated tryptophan metabolites or derivatives thereof or pharmaceutically acceptable salts thereof.

2. A method of downregulating B cell functioning in a mammal, said method comprising administering to said mammal an effective amount of one or more IDO-mediated tryptophan metabolites or derivatives thereof or pharmaceutically acceptable salts thereof.

3. The method according to claim 1, wherein the IDO-mediated tryptophan metabolite or derivative thereof is a compound of formula (I): 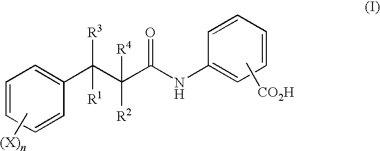
wherein each of R¹ and R² is independently selected from a hydrogen atom or a C₁-C₄ alkyl group, R³ and R⁴ are each hydrogen atoms or together form another chemical bond, each X is independently selected from a hydroxyl group, a halogen atom, a C₁-C₄ alkyl group or a C₁-C₄ alkoxy group, or when two X groups are alky or alkoxy groups, they may be connected together to form a ring, and n is an integer from 1 to 3 or a pharmaceutically acceptable salt thereof.

4. The method according to claim 3, wherein the IDO-mediated tryptophan metabolite or derivative thereof is a compound of formula (II): 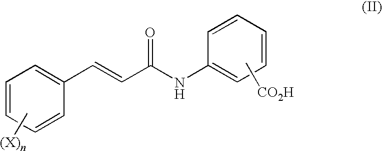
wherein X and n are as defined in claim 3.

5. The method according to claim 4, wherein the compound of formula (II) is selected from:
2-[[3-(2-methylphenyl)-1-oxo-2-propenyl]amino]benzoic acid;
2-[[3-(3-methylphenyl)-1-oxo-2-propenyl]amino]benzoic acid;
2-[[3-(4-methylphenyl)-1-oxo-2-propenyl]amino]benzoic acid;
2-[[3-(2-ethylphenyl)-1-oxo-2-propenyl]amino]benzoic acid;
2-[[3-(3-ethylphenyl)-1-oxo-2-propenyl]amino]benzoic acid;
2-[[3-(4-ethylphenyl)-1-oxo-2-propenyl]amino]benzoic acid;
2-[[3-(2-propylphenyl)-1-oxo-2-propenyl]amino]benzoic acid;
2-[[3-(3-propylphenyl)-1-oxo-2-propenyl]amino]benzoic acid;
2-[[3-(4-propylphenyl)-1-oxo-2-propenyl]amino]benzoic acid;
2-[[3-(2-hydroxyphenyl)-1-oxo-2-propenyl]amino]benzoic acid;
2-[[3-(3-hydroxyphenyl)-1-oxo-2-propenyl]amino]benzoic acid;
2-[[3-(4-hydroxyphenyl)-1-oxo-2-propenyl]amino]benzoic acid;
2-[[3-(2-chlorophenyl)-1-oxo-2-propenyl]amino]benzoic acid;
2-[[3-(3-chlorophenyl)-1-oxo-2-propenyl]amino]benzoic acid;
2-[[3-(4-chlorophenyl)-1-oxo-2-propenyl]amino]benzoic acid;
2-[[3-(2-fluorophenyl)-1-oxo-2-propenyl]amino]benzoic acid;
2-[[3-(3-fluorophenyl)-1-oxo-2-propenyl]amino]benzoic acid;
2-[[3-(4-fluorophenyl)-1-oxo-2-propenyl]amino]benzoic acid;
2-[[3-(2-bromophenyl)-1-oxo-2-propenyl]amino]benzoic acid;
2-[[3-(3-bromophenyl)-1-oxo-2-propenyl]amino]benzoic acid;
2-[[3-(4-bromophenyl)-1-oxo-2-propenyl]amino]benzoic acid;
2-[[3-(2,3-dimethoxyphenyl)-1-oxo-2-propenyl]amino]benzoic acid;
2-[[3-(3,4-dimethoxyphenyl)-1-oxo-2-propenyl]amino]benzoic acid;
2-[[3-(2,4-dimethoxyphenyl)-1-oxo-2-propenyl]amino]benzoic acid;
2-[[3-(2,3-dimethylphenyl)-1-oxo-2-propenyl]amino]benzoic acid;
2-[[3-(3,4-dimethylpheny I)-1-oxo-2-propenyl]amino]benzoic acid;
2-[[3-(2,4-dimethylphenyl)-1-oxo-2-propenyl]amino]benzoic acid;
2-[[3-(2,3-diethoxyphenyl)-1-oxo-2-propenyl]amino]benzoic acid;
2-[[3-(3,4-diethoxyphenyl)-1-oxo-2-propenyl]amino]benzoic acid;
2-[[3-(2,4-diethoxyphenyl)-1-oxo-2-propenyl]amino]benzoic acid;
2-[[3-(2,3-dipropoxyphenyl)-1-oxo-2-propenyl]amino]benzoic acid;
2-[[3-(3,4-dipropoxyphenyl)-1-oxo-2-propenyl]amino]benzoic acid;
2-[[3-(2,4-dipropoxyphenyl)-1-oxo-2-propenyl]amino]benzoic acid;
2-[[3-(2,3-diethylphenyl)-1-oxo-2-propenyl]amino]benzoic acid;
2-[[3-(3,4-diethylphenyl)-1-oxo-2-propenyl]amino]benzoic acid;
2-[[3-(2,4-diethylphenyl)-1-oxo-2-propenyl]amino]benzoic acid;
2-[[3-(2,3-dipropylphenyl)-1-oxo-2-propenyl]amino]benzoic acid;
2-[[3-(3,4-dipropylphenyl)-1-oxo-2-propenyl]amino]benzoic acid;
2-[[3-(2,4-dipropylphenyl)-1-oxo-2-propenyl]amino]benzoic acid;
2-[[3-(2-methoxy-3-methylphenyl)-1-oxo-2-propenyl]amino]benzoic acid;
2-[[3-(3-methoxy-4-methylphenyl)-1-oxo-2-propenyl]amino]benzoic acid;
2-[[3-(2-methoxy-3-methylphenyl)-1-oxo-2-propenyl]amino]benzoic acid;
2-[[3-(2-methoxy-4-methylphenyl)-1-oxo-2-propenyl]amino]benzoic acid;
2-[[3-(2-methoxy-3-chlorophenyl)-1-oxo-2-propenyl]amino]benzoic acid;
2-[[3-(3-methoxy-4-chlorophenyl)-1-oxo-2-propenyl]amino]benzoic acid;
2-[[3-(2-methoxy-3-chlorophenyl)-1-oxo-2-propenyl]amino]benzoic acid;
2-[[3-(2-methoxy-4-chlorophenyl)-1-oxo-2-propenyl]amino]benzoic acid;
2-[[3-(2-methoxy-3-hydroxyphenyl)-1-oxo-2-propenyl]amino]benzoic acid;
2-[[3-(3-methoxy-4-hydroxyphenyl)-1-oxo-2-propenyl]amino]benzoic acid;
2-[[3-(2-methoxy-3-hydroxyphenyl)-1-oxo-2-propenyl]amino]benzoic acid;
2-[[3-(2-methoxy-4-hydroxyphenyl)-1-oxo-2-propenyl]amino]benzoic acid;
2-[[3-(3,4-trimethylenephenyl)-1-oxo-2-propenyl]amino]benzoic acid;
2-[[3-(2,3-trimethylenephenyl)-1-oxo-2-propenyl]amino]benzoic acid;
2-[[3-(3,4-methylenedioxyphenyl)-1-oxo-2-propenyl]amino]benzoic acid; and
2-[[3-(3,4-ethylenedioxyphenyl)-1-oxo-2-propenyl]amino]benzoic acid.

6. The method according to claim 4, wherein the compound of formula (II) is 2-[[3-(3,4-dimethoxyphenyl)-1-oxo-2-propenyl]amino]benzoic acid (Tranilast).

7. The method according to claim 1, wherein the IDO-mediated tryptophan metabolite or derivative thereof is a compound of formula (III): 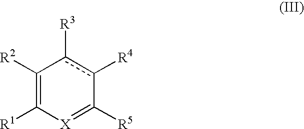
wherein
X is selected from N and CR⁶; [symbol]
represents a single or double bond;
R¹ is selected from H, C₁₋₄ alkyl, OH, C₁₋₄ alkoxy, halo, CO₂H and CO₂C₁₋₄ alkyl;
R² is selected from H, C₁₋₄ alkyl, OH, C₁₋₄ alkoxy, halo, or R¹ and R² together form an optionally substituted fused phenyl ring;
R³ is selected from H, C₁₋₄ alkyl, OH, C₁₋₄ alkoxy and halo;
R⁴ is selected from H, C₁₋₄ alkyl, C₂₋₄ alkenyl, OH, C₁₋₄ alkoxy, CO₂H, CO₂C₁₋₄ alkyl and 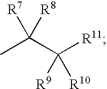
R⁵ is selected from C₁₋₄ alkyl, OH, C₁₋₄ alkoxy, halo, CO₂H, CO₂C₁₋₄ alkyl, NH₂ and NHR¹²;
R⁶ is selected from H, C₁₋₄ alkyl, OH and C₁₋₄ alkoxy;
R⁷, R⁸, R⁹ and R¹⁰ are each independently H and C₁₋₄ alkyl or R⁷ and R⁸ together form an oxo group or R⁷ and R⁹ form a bond;
R¹¹ is selected from CH(CO₂H)NH₂, CH(CO₂C₁₋₄alkyl)NH₂, C(O)CO₂H, C(O)CO₂C₁₋₄alkyl, C(O)H, CO₂H, CO₂C₁₋₄alkyl, C(O)NH₂, C(O)NHR¹³, CH₂NH₂, CH₂NHC₁₋₄alkyl and CH₂N(C₁₋₄alkyl)₂;
R¹² is selected from H, C₁₋₄alkyl and C(O)H; and
R¹³ is H, C₁₋₄alkyl and optionally substituted phenyl, wherein optionally substituted phenyl is optionally substituted with one or more, C₁₋₄alkyl, OH, C₁₋₄alkoxy, CO₂H, CO₂C₁₋₄alkyl, halo, NH₂, NHC₁₋₄alkyl and N(C₁₋₄alkyl)₂ or a pharmaceutically acceptable salt thereof.

8. The method according to claim 7, wherein the compound of formula (III) is 3-hydroxykynurenic acid, 3-hydroxyanthranilic acid, picolinic acid or quinolinic acid.

9. The method according to claim 1, wherein said B cell functioning is B cell proliferation.

10. The method according to claim 1, wherein said B cell functioning is antibody production.

11. A method for the treatment and/or prophylaxis of a condition characterized by aberrant or unwanted B cell functioning in a mammal, said method comprising administering to said mammal an effective amount of one or more IDO-mediated tryptophan metabolites or derivatives thereof or pharmaceutically acceptable salts thereof for a time and under conditions sufficient to downregulate said B cell functioning.

12. The method according to claim 11, wherein the IDO-mediated tryptophan metabolite or derivative thereof is a compound of formula (I): 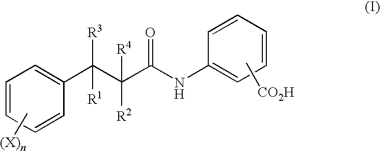
wherein each of R¹ and R² is independently selected from a hydrogen atom or a C₁-C₄alkyl group, R³ and R⁴ are each hydrogen atoms or together form another chemical bond, each X is independently selected from a hydroxyl group, a halogen atom, a C₁-C₄alkyl group or a C₁-C₄alkoxy group, or when two X groups are alkyl or alkoxy groups, they may be connected together to form a ring, and n is an integer from 1 to 3 or a pharmaceutically acceptable salt thereof.

13. The method according to claim 12, wherein the IDO-mediated tryptophan metabolite or derivative thereof is a compound of formula (II): 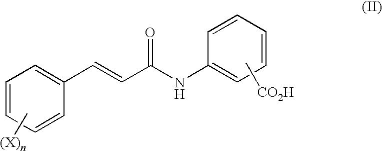
wherein X and n are as defined in claim 12.

14. The method according to claim 13, wherein the compound of formula (II) is selected from:
2-[[3-(2-methylphenyl)-1-oxo-2-propenyl]amino]benzoic acid;
2-[[3-(3-methylphenyl)-1-oxo-2-propenyl]amino]benzoic acid;
2-[[3-(4-methylphenyl)-1-oxo-2-propenyl]amino]benzoic acid;
2-[[3-(2-ethylphenyl)-1-oxo-2-propenyl]amino]benzoic acid;
2-[[3-(3-ethylphenyl)-1-oxo-2-propenyl]amino]benzoic acid;
2-[[3-(4-ethylphenyl)-1-oxo-2-propenyl]amino]benzoic acid;
2-[[3-(2-propylphenyl)-1-oxo-2-propenyl]amino]benzoic acid;
2-[[3-(3-propylphenyl)-1-oxo-2-propenyl]amino]benzoic acid;
2-[[3-(4-propylphenyl)-1-oxo-2-propenyl]amino]benzoic acid;
2-[[3-(2-hydroxyphenyl)-1-oxo-2-propenyl]amino]benzoic acid;
2-[[3-(3-hydroxyphenyl)-1-oxo-2-propenyl]amino]benzoic acid;
2-[[3-(4-hydroxyphenyl)-1-oxo-2-propenyl]amino]benzoic acid;
2-[[3-(2-chlorophenyl)-1-oxo-2-propenyl]amino]benzoic acid;
2-[[3-(3-chlorophenyl)-1-oxo-2-propenyl]amino]benzoic acid;
2-[[3-(4-chlorophenyl)-1-oxo-2-propenyl]amino]benzoic acid;
2-[[3-(2-fluorophenyl)-1-oxo-2-propenyl]amino]benzoic acid;
2-[[3-(3-fluorophenyl)-1-oxo-2-propenyl]amino]benzoic acid;
2-[[3-(4-fluorophenyl)-1-oxo-2-propenyl]amino]benzoic acid;
2-[[3-(2-bromophenyl)-1-oxo-2-propenyl]amino]benzoic acid;
2-[[3-(3-bromophenyl)-1-oxo-2-propenyl]amino]benzoic acid;
2-[[3-(4-bromophenyl)-1-oxo-2-propenyl]amino]benzoic acid;
2-[[3-(2,3-dimethoxyphenyl)-1-oxo-2-propenyl]amino]benzoic acid;
2-[[3-(3,4-dimethoxyphenyl)-1-oxo-2-propenyl]amino]benzoic acid;
2-[[3-(2,4-dimethoxyphenyl)-1-oxo-2-propenyl]amino]benzoic acid;
2-[[3-(2,3-dimethylphenyl)-1-oxo-2-propenyl]amino]benzoic acid;
2-[[3-(3,4-dimethylphenyl)-1-oxo-2-propenyl]amino]benzoic acid;
2-[[3-(2,4-dimethyrphenyl)-1-oxo-2-propenyl]amino]benzoic acid;
2-[[3-(2,3-diethoxyphenyl)-1-oxo-2-propenyl]amino]benzoic acid;
2-[[3-(3,4-diethoxyphenyl)-1-oxo-2-propenyl]amino]benzoic acid;
2-[[3-(2,4-diethoxyphenyl)-1-oxo-2-propenyl]amino]benzoic acid;
2-[[3-(2,3-dipropoxyphenyl)-1-oxo-2-propenyl]amino]benzoic acid;
2-[[3-(3,4-dipropoxyphenyl)-1-oxo-2-propenyl]amino]benzoic acid;
2-[[3-(2,4-dipropoxyphenyl)-1-oxo-2-propenyl]amino]benzoic acid;
2-[[3-(2,3-diethylphenyl)-1-oxo-2-propenyl]amino]benzoic acid;
2-[[3-(3,4-diethylphenyl)-1-oxo-2-propenyl]amino]benzoic acid;
2-[[3-(2,4-diethylphenyl)-1-oxo-2-propenyl]amino]benzoic acid;
2-[[3-(2,3-dipropylphenyl)-1-oxo-2-propenyl]amino]benzoic acid;
2-[[3-(3,4-dipropylphenyl)-1-oxo-2-propenyl]amino]benzoic acid;
2-[[3-(2,4-dipropylphenyl)-1-oxo-2-propenyl]amino]benzoic acid;
2-[[3-(2-methoxy-3-methylphenyl)-1-oxo-2-propenyl]amino]benzoic acid;
2-[[3-(3-methoxy-4-methylphenyl)-1-oxo-2-propenyl]amino]benzoic acid;
2-[[3-(2-methoxy-3-methylphenyl)-1-oxo-2-propenyl]amino]benzoic acid;
2-[[3-(2-methoxy-4-methylphenyl)-1-oxo-2-propenyl]amino]benzoic acid;
2-[[3-(2-methoxy-3-chlorophenyl)-1-oxo-2-propenyl]amino]benzoic acid;
2-[[3-(3-methoxy-4-chlorophenyl)-1-oxo-2-propenyl]amino]benzoic acid;
2-[[3-(2-methoxy-3-chlorophenyl)-1-oxo-2-propenyl]amino]benzoic acid;
2-[[3-(2-methoxy-4-chlorophenyl)-1-oxo-2-propenyl]amino]benzoic acid;
2-[[3-(2-methoxy-3-hydroxyphenyl)-1-oxo-2-propenyl]amino]benzoic acid;
2-[[3-(3-methoxy-4-hydroxyphenyl)-1-oxo-2-propenyl]amino]benzoic acid;
2-[[3-(2-methoxy-3-hydroxyphenyl)-1-oxo-2-propenyl]amino]benzoic acid;
2-[[3-(2-methoxy-4-hydroxyphenyl)-1-oxo-2-propenyl]amino]benzoic acid;
2-[[3-(3,4-trimethylenephenyl)-1-oxo-2-propenyl]amino]benzoic acid;
2-[[3-(2,3-trimethylenephenyl)-1-oxo-2-propenyl]amino]benzoic acid;
2-[[3-(3,4-methylenedioxyphenyl)-1-oxo-2-propenyl]amino]benzoic acid; and
2-[[3-(3,4-ethylenedioxyphenyl)-1-oxo-2-propenyl]amino]benzoic acid.

15. The method according to claim 13, wherein the compound of formula (II) is 2-[[3-(3,4-dimethoxyphenyl)-1-oxo-2-propenyl]amino]benzoic acid (Tranilast).

16. The method according to claim 11, wherein the IDO-mediated tryptophan metabolite or derivative thereof is a compound of formula (III): 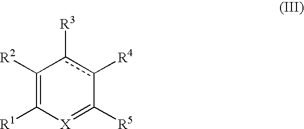
wherein
X is selected from N and CR⁶; [symbol]
represents a single or double bond;
R¹ is selected from H, C₁₋₄alkyl, OH, C₁₋₄alkoxy, halo, CO₂H and CO₂C₁₋₄alkyl;
R² is selected from H, C₁₋₄alkyl, OH, C₁₋₄alkoxy, halo, or R¹ and R² together form an optionally substituted fused phenyl ring;
R³ is selected from H, C₁₋₄alkyl, OH, C₁₋₄alkoxy and halo;
R⁴ is selected from H, C₁₋₄alkyl, C₂₋₄alkenyl, OH, C₁₋₄alkoxy, CO₂H, CO₂C₁₋₄alkyl
and 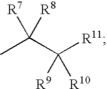
R⁵ is selected from C₁₋₄alkyl, OH, C₁₋₄alkoxy, halo, CO₂H, CO₂C₁₋₄alkyl, NH₂ and NHR¹²;
R⁶ is selected from H, C₁₋₄alkyl, OH and C₁₋₄alkoxy;
R⁷, R⁸, R⁹ and R¹⁰ are each independently H and C₁₋₄alkyl or R⁷ and R⁸ together form an oxo group or R⁷ and R⁹ form a bond;
R¹¹ is selected from CH(CO₂H)NH₂, CH(CO₂C₁₋₄alkyl)NH₂, C(O)CO₂H, C(O)CO₂C₁₋₄alkyl, C(O)H, CO₂H, CO₂C₁₋₄alkyl, C(O)NH₂, C(O)NHR¹³, CH₂NH₂, CH₂NHC₁₋₄alkyl and CH₂N(C₁₋₄alkyl)₂;
R¹² is selected from H, C₁₋₄alkyl and C(O)H; and
R¹³ is H, C₁₋₄alkyl and optionally substituted phenyl, wherein optionally substituted phenyl is optionally substituted with one or more, C₁₋₄alkyl, OH, C₁₋₄alkoxy, CO₂H, CO₂C₁₋₄alkyl, halo, NH₂, NHC₁₋₄alkyl and N(C₁₋₄alkyl)₂ or a pharmaceutically acceptable salt thereof.

17. The method according to claim 16, wherein the compound of formula (III) is 3-hydroxykynurenic acid, 3-hydroxyanthranilic acid, picolinic acid or quinolinic acid.

18. The method according to claim 11, wherein said B cell functioning is B cell proliferation or antibody production.

19. (canceled)

20. The method according to claim 18, wherein said condition is an autoimmune condition.

21. The method according to claim 20, wherein said autoimmune condition is rheumatoid arthritis, multiple sclerosis, systemic Lupus Erythamatosus, Crohn's disease, inflammatory bowel disease, type I diabetes, psoriasis, Sjogren's syndrome, Graves' disease, autoimmune thyroiditis, systemic sclerosis, chronic immune thrombocytopenic purpura, autoimmune haemolytic anaemia, autoimmune polyneuropathy, Wegener's granulomatosis, cold agglutinin disease associated with indolent lymphoma, idiopathic membranous neuropathy, type II mixed cryoglobulinaemia, acquired factor VIII inhibitors, fludarabine-associated immune thrombocytopenic purpura, refractory dermatomyositis, pemphigus vulgaris or myasthenia gravis.

22. The method according to claim 18, wherein said condition is a non-autoimmune condition.

23. The method according to claim 22 wherein said non-autoimmune condition is graft versus host disease, acute or chronic transplant rejection, septic shock, insulin resistance, apoptotic conditions or neoplastic conditions.

24. The method according to claim 23 wherein said neoplastic condition is a B cell neoplasia.

25. The method according to claim 24 wherein said B cell neoplasia is B-chronic lymphocytic leukaemia, indolent and follicular lymphoma, mantle cell lymphoma, small lymphocytic lymphoma, multiple myeloma, primary cutaneous B cell lymphoma, acute lymphocytic leukaemia, Burkitt's lymphoma, HIV-associated lymphoma, primary CNS lymphoma, post-transplant lymphoproliferative disorder or Hodgkin's disease.

26-54. (canceled)
